SEQUENCE LISTING (5 sequences)

SEQ ID NO: 1 - protein, 365 aa, synthetic construct
MHHHHHHGGGGSVLVKNEPQLKTPKNVILLISDGAGLSQISSTFYFKEGTPNYTQFKNIGLIKTSSSREDVTDSASGATAFSCGIKTYNAAIGVADDSTAVKSIVEIAALNNIKTGVVATSSITHATPASFYAHALNRGLEEEIAMDMTESDLDFFAGGGLNYFTKRKDKKDVLAILKGNQFTINTTGLTDFSSIASNRKMGFLLADEAMPTMEKGRGNFLSAATDLAIQFLSKDNSAFFIMSEGSQIDWGGHANNASYLISEINDFDDAIGTALAFAKKDGNTLVIVTSDHETGGFTLAAKKNKREDGSEYSDYTEIGPTFSTGGHSATLIPVFAYGPGSEEFIGIYENNEIFHKILKVTKWNQ

SEQ ID NO: 2 - protein, 8 aa, synthetic construct
HRHRHRHR

SEQ ID NO: 3 - protein, 5 aa, synthetic construct
GGGGS

SEQ ID NO: 4 - protein, 9 aa, synthetic construct
HRHRHRHRP

SEQ ID NO: 5 - protein, 6 aa, synthetic construct
HHHHHH

(19) United States
(12) Patent Application Publication
(10) Pub. No.: US 2025/0243525 A1
(43) Pub. Date: Jul. 31, 2025

(54) BIOCATALYTIC MANUFACTURE OF SUGAR NUCLEOTIDES

(71) Applicant: ZYMtronix Catalytic Systems, Inc., Ithaca, NY (US)

(72) Inventors: Alexander Chris Hoepker, Ithaca, NY (US); Megha Cila, Chapel Hill, NC (US); Fabian Giska, Morrisville, NC (US)

(73) Assignee: ZYMtronix Catalytic Systems, Inc., Ithaca, NY (US)

(21) Appl. No.: 18/878,249

(22) PCT Filed: Jun. 23, 2023
(86) PCT No.: PCT/US2023/026163

§ 371 Date: Dec. 23, 2024

Related U.S. Application Data
(60) Provisional application No. 63/355,450, filed on Jun. 24, 2022.

(51) Int. Cl. C12P 19/40; C12N 9/12; C12N 9/16; C12N 11/14

(52) U.S. Cl. CPC C12P 19/40; C12N 9/12; C12N 9/16; C12N 11/14; C12Y 207/07064; C12Y 301/03001; C12Y 306/01001

(57) ABSTRACT

The invention provides the production of sugar-nucleotides and the isolation of sugar-nucleotides. In some embodiments the production or isolation are accomplished under acidic conditions. The production is a cell-free synthesis using enzymes, including immobilized enzymes. They may be accomplished using a one-pot reaction protocol. The synthesis may be used as a highly customizable and highly efficient cell-free manufacturing process. In some embodiments, the sugar-nucleotides are used to prepare UDP-Gal, lactose derivatives, and human milk oligosaccharides (HMOs).

### CROSS REFERENCE TO RELATED APPLICATIONS

This application claims priority under 35 U.S.C. § 119(e) to U.S. Provisional Application No. 63/355,450, filed Jun. 24, 2022, and incorporated herein by reference in its entirety.

### FIELD OF INVENTION

The invention provides compositions and methods for manufacturing sugar-nucleotide compounds and purification thereof. The methods are highly customizable, scalable, and efficient manufacturing processes.

### BACKGROUND OF THE INVENTION

Cell free systems have been of interest in biomanufacturing, but there are challenges (M. P. Cordoso Marques et al., Adv. Biochem. Eng. Biotechnol., https://doi.org/10.1007/10_2020_160; C. You, Adv. Biochem. Engl, DOI: 10.1007/10_2012_159 (2012)); C. H. Hokke, et al., Glycoconjugate Journal 13.4:687-692 (1996); A. Zervosen and L. Elling, “A Novel Three-Enzyme Reaction Cycle for the Synthesis of N-Acetyllactosamine with in Situ Regeneration of Uridine 5′-Diphosphate Glucose and Uridine 5′-Diphosphate Galactose” J. Am. Chem. Soc. 118.8:1836-1840 (1996). The foregoing are incorporated by reference herein in their entirety.

Thus, the art seeks an economical and efficient way to produce glycans, including human milk oligosaccharides (HMOs). These include, but are not limited to, glycans of two sugar units or larger or four sugar units or larger. This would overcome major hurdles in advancing these glycans and HMOs for probiotic, prophylactic, and therapeutic purposes. The economical and efficient HMO production would be an important advance for global infant nutrition and disease prevention. Importantly, the invention provides enhanced production efficiency of sugars and complex carbohydrates while lowering costs. This is a vast improvement over the art because it improves the accessibility and affordability of these molecules.

Glycans are complex carbohydrate structures that are the predominant molecules on the cell surface and serve as the first point of contact between cells, the with the extracellular matrix, and with pathogens. Glycans are involved in a number of biological processes such as cell-to cell-interactions. There is great interest in improving the accessibility and affordability of these molecules for research, preclinical, and commercial applications.

Activated sugars are often used as research reagents to enable uses of glycans or the discovery of new glycans. The use of glycans and activated sugars is anew technology that could have wide applications. Galactose-UDP is an activated sugar and a precursor building block for glycans in living organisms. T. Jaroentomeechai, et al., Frontiers in Chemistry, 8:645, (2020); M. Lemmerer, et al., Advanced Synthesis & Catalysis 358, 3113-3122 (2016); Y. Zou, et al. Carbohydrate Res. 373:76-81 (2013); EP3819381A1; CN113265434A; G. Zhao, et al., Nat. Protoc. 5:636-646 (2010); A. Zervosen, et al., J. Mol. Catal. B: Enzym. 5:25-28 (1998); H. Tanaka, et al., Angew. Chem. 124:11699-11702 (2012), Angew. Chem. Int. Ed 51:11531-11534 (2012); S. Amann et al., Carbohydr. Res., 335:23-32 (2001); Y. Zhai, et al., Biotechnol. Lett., 34:1321-1326 (2012); R. Marquardt, et al., U.S. Pat. No. 5,866,378; E. Baroja-Femandez, et al., Proc. Natl. Acad. Sci. USA 109:321-326 (2012). The foregoing are incorporated by reference herein in their entirety

Thus, the art seeks an economical and efficient way to produce nucleotide activated sugars, including, but not limited to, nucleotide-galactose compounds and UDP-galactose (https://pubchem.ncbi.nlm.nih.gov/compound/UDP-alpha-D-galactose, https://www.sigmaaldrich.com/US/en/product/sigma/u4500). This would overcome major hurdles in advancing the production of nucleotide sugars such as UDP-Galactose, galactosylated glycans, and HMOs for probiotic, prophylactic, and therapeutic purposes. The economical and efficient production of nucleotide sugars and HMOs would be an important advance for global infant nutrition, drug development, disease treatment, prophylaxis, and prevention.

### SUMMARY OF THE INVENTION

The present invention provides processes for preparing sugar-nucleotide compounds and processes for purifying the same. The processes employ phosphatases that hydrolyze nucleotides in the presence of sugar-nucleotides under conditions that do not hydrolyze sugar-nucleotides. In some embodiments, the invention provides processes for preparing nucleotide-galactose compounds starting from galactose. The processes are cost effective and industrially scalable to produce UDP-galactose and other galactose compounds by increasing feed concentrations, minimizing feed costs, lowering the costs of operations, and in some embodiments adopting the process for cGMP production. In one embodiment, continuous manufacturing with immobilized enzymes provides a dramatic reduction in operational costs and allows for easier adoption to a cGMP process.

The invention provides a simple, cost-effective purification process that does not rely on chromatographic methods. The sugar-nucleotides are useful in synthesizing sugar-containing compounds and the sugar-nucleotides are useful in methods of treatment, prevention, and prophylaxis. This includes, but is not limited to, cancer treatments and triggering immune responses, including but not limited to, in humans.

In one embodiment, this invention provides a process for converting adenosine diphosphate to adenosine, comprising the step of contacting a first composition comprising adenosine diphosphate and a sugar-nucleotide with a phosphatase at an acidic pH to convert the first composition to a second composition comprising adenosine, phosphate, and the sugar-nucleotide. Another embodiment further comprises the initial step of generating the first composition by contacting a sugar, a nucleotide triphosphate, and adenosine triphosphate with a kinase, a nucleotide sugar pyrophosphorylase, and an inorganic pyrophosphorylase.

In certain embodiments, phosphatase is an alkaline or acidic phosphatase. In some embodiments, the alkaline phosphatase is an Antarctic phosphatase. In some embodiments, the Antarctic phosphatase is a bacterial Antarctic phosphatase. In certain embodiments, the bacterial Antarctic phosphatase has Tab5 activity and has at least a 90% sequence identity to SEQ ID NO:1. In a preferred embodiment, the Tab5 has the sequence of SEQ ID NO:1.

Enzymes that have sequence variations compared to SEQ ID NO:1 but function as a phosphatase are included within this invention. In some embodiments, the invention utilizes enzymes having Tab5 activity and at least a 90% sequence identity to SEQ ID NO:1. The invention contemplates enzymes that include, but are not limited to, enzymes that have similar phosphatase activity as Tab5 but that have a different sequence than SEQ ID NO:1. In some embodiments, a Tab5-related phosphatase may be obtained by, for example, protein engineering.

The certain embodiments, the acidic pH is 6.9 or below. In other embodiments, the acidic pH is 5.0 to pH 6.9.

Some embodiments further comprise a processing step to separate the sugar-nucleotide and adenosine. In certain embodiments, the processing step is selective precipitation of the sugar-nucleotide in the presence of an antisolvent or a water-miscible solvent. Some embodiments further comprise the steps of concentrating the second composition to obtain a concentrate, adding a salt to the concentrate to obtain a salted concentrate, adding an antisolvent or a water miscible solvent to the salted concentrate to provide a precipitate solution, separating the precipitate solution to obtain a filtrate and a precipitate, drying the precipitate to obtain the sugar-nucleotide. Certain embodiments further comprise an initial step of removing the kinase, the nucleotide sugar pyrophosphorylase, and the inorganic pyrophosphorylase to provide a filtered output composition. In some embodiments, the filtering removes the phosphatase, the kinase, the nucleotide sugar pyrophosphorylase, and the inorganic pyrophosphorylase by a filtration or an adsorption method. In certain embodiments, the filtration is ultrafiltration, tangential flow filtration, or diafiltration. In some embodiments, the concentrating to obtain the concentrate is by a factor of about 2 to about 100. In some embodiments, the concentrating is by tangential flow nanofiltration.

In some embodiments, the salt is an ammonium salt, a lithium salt, a sodium salt, or a potassium salt. In certain embodiments, the sodium salt is sodium acetate or the potassium salt is potassium acetate. In some embodiments, the sodium acetate is present in about 10 mM to about 200 mM. In other embodiments, the potassium acetate is present in about 10 mM to about 200 mM.

In some embodiments, the water miscible solvent is an alcohol. In certain embodiments, the alcohol is ethanol, methanol, or isopropanol. In preferred embodiments, the alcohol is ethanol. In some embodiments, the water miscible solvent has a solvent volume of 2, 3, 4, 5, 6, 7, 8, 9, or 10 volumes of solvent relative to the salted concentrate volume. Certain embodiments of this invention further comprise an incubation step, wherein the water miscible solvent and the salted concentrate are incubated. In some embodiments the incubation step is at a temperature of about −20° C. to about 4° C. In some embodiments, the incubation step is at an incubation time of about 2 hours to about 24 hours.

In some embodiments, the separating is by a physical separation method. In certain embodiments, the physical separation method is filtration, centrifugation, or decanting.

In some embodiments, employing a drying step, the drying is by spray drying, freeze drying, vacuum drying, or evaporative drying. In a more specific embodiment, the drying is by spray drying.

In one embodiment, this invention provides a process for preparing UDP-galactose comprising the step of: contacting galactose, UTP, ATP, a galactose kinase, a UDP-sugar pyrophosphorylase, and an inorganic pyrophosphatase to prepare UDP-galactose.

In one embodiment, this invention provides a process for preparing UDP-galactose comprising the step of contacting galactose, UTP, ATP, BiGalK, BlUSP, PmPpa to prepare UDP-galactose.

In one embodiment, this invention provides a process for preparing UDP-galactose comprising the steps of:
a. contacting galactose, UTP, ATP, BiGalK, BlUSP, PmPpa to produce UDP-galactose in a reaction mixture and an alkaline phosphatase; and
b. purifying the UDP-galactose by filtration.

In certain embodiments, the saccharide is glucose, galactose, fructose, rhamnose, mannose, ribose, xylose, arabinose, fucose, apiose, glucuronate, galacturonate, N-acetylglucosamine, N-acetylgalactosamine, or N-acetylneuraminic acid.

In certain embodiments, the saccharide is D-Glucose, D-galactose, D-fructose, L-rhamnose, D-mannose, D-ribose, D-xylose, L-arabinose, D-xylose, L-fucose, D-apiose, D-glucuronate, D-galacturonate, N-acetyl-D-glucosamine, N-acetyl-D-galactosamine, or N-acetylneuraminic acid.

In other embodiments, the nucleotide is uridine 5′-diphosphate (UDP) and the sugar is D-glucose, D-galactose, D-fructose, L-rhamnose, D-mannose, D-ribose, D-xylose, L-arabinose, D-xylose, L-fucose, D-apiose, D-glucuronate, D-galacturonate, N-acetyl-D-glucosamine, or N-acetyl-D-galactosamine

In other embodiments, the nucleoside is adenosine 5′-diphosphate (ADP) and the sugar is D-glucose, D-galactose, D-fructose, D-mannose, D-ribose, L-arabinose, D-xylose, or D-glucuronate.

In other embodiments, the nucleotide is guanosine 5′-diphosphate (GDP) and the sugar is D-Glucose, D-galactose, D-mannose, L-rhamnose, D-ribose, D-xylose, L-arabinose, or L-fucose.

In other embodiments, the nucleotide is cytidine 5-monosphate (CMP) and the sugar is N-acetylneuraminic acid.

In other embodiments, the nucleotide is thymidine 5′-diphosphate (TDP) and the sugar is D-Glucose or D-galacturonate.

In certain embodiments, the phosphatase, the kinase, the nucleotide sugar pyrophosphorylase, or the inorganic pyrophosphorylase is a free enzyme. In other embodiments, the phosphatase, the kinase, the nucleotide sugar pyrophosphorylase, or the inorganic pyrophosphorylase is an immobilized enzyme. In embodiments employing an immobilized enzyme, the immobilization is via a N-terminal tag or a C-terminal tag. In such embodiments, the N-terminal tag or the C-terminal tag is polvarginine, polylysine, or histidine-arginine repeats.

In some embodiments, the sugar-nucleotide pyrophosphorylase is a uridine 5′-diphosphate-sugar-pyrophosphorylase (UDP-sugar pyrophosphorylase). In other embodiments, the nucleotide sugar pyrophosphorylase is a UDP-sugar pyrophosphorylase (USP) or a UTP-glucose 1-phosphate uridylyltransferase (GalU). In other embodiments, the UDP-sugar pyrophosphorylase is BlUSP, EcUSP, or SpUSP. In specific embodiments, the UDP-sugar pyrophosphorylase is BlUSP. In other embodiments, the UTP-Glucose 1-phosphate uridylvltransferase is SpGalU.

In some embodiments, the kinase is a galactokinase. In certain embodiments, galactokinase is galactokinase from Bifidobacterium infantis (BiGalK), galactokinase from Streptococcus pneumoniae (SpGalK), galactokinase from Escherichia coli (EcGalK), or galactokinase from Leminorella grimontii (LgGalK). In specific embodiments, the galactokinase is from Bifidobacterium infantis (BiGalK). L. Li, et al. Carbohydrate Research 355:35-39 (2012).

In certain embodiments, the inorganic pyrophosphatase is inorganic pyrophosphatase from Pasteurella multocida (PmPpa) or inorganic pyrophosphatase from Escherichia coli (EcPpa). In specific embodiments, the inorganic pyrophosphatase is from Pasteurella multocida (PmPpa).

In some embodiments, an immobilized enzyme is immobilized via an iron oxide material. In certain embodiments, the iron oxide material is hematite, magnetite, or strontium ferrite. In certain embodiments, the immobilized enzyme is a Type A scaffolded BNC. In other embodiments, the immobilized enzyme is a Type B scaffolded BNC.

In certain embodiments, one or more enzymes are immobilized within bionanocatalysts (BNCs) which in turn are embedded within scaffolds. Bionanocatalysts (BNCs) according to this invention comprise an enzyme self-assembled with magnetic nanoparticles (MNPs). The BNCs self-assemble with the scaffolds. In certain embodiments, each enzyme is immobilized within the BNC.

In certain embodiments, the scaffolded BNCs are inside of modular flow cells for flow manufacturing. In certain embodiments, the invention provides continuous flow processing where each step of synthesis is conducted in modules. In production mode, these modules contain full systems of enzymes—sugar activation and sugar transfer—for specifically building glycans. In some embodiments, the glycans are oligosaccharides.

In some embodiments, the scaffolds comprise magnetic metal oxides. In some embodiments, the scaffolds are high magnetism and high porosity composite blends of thermoplastics comprising magnetic particles that form powders. They may be single-layered or multiple-layered materials that hold the BNCs. Such designed objects may be produced using 3D printing by sintering composite magnetic powders. In some embodiments, Selective Laser Sintering (SLS) is used. The modular flow cells may be mixed and matched for a highly customizable and highly efficient manufacturing process. In preferred embodiments, human milk oligosaccharides (HMOs) are produced.

Thus, the invention provides cell-free productions of sugar-nucleotides with combinatorial bionanocatalysts (BNCs) organized in sequential modules.

In preferred embodiments, the sugar-nucleotide is UDP-galactose. In certain embodiments, the UDP-galactose has about or at least 97%, about or at least 98%, or about or at least 99% purity.

In certain embodiments, a process of this invention occurs in a single reaction vessel. In some embodiments, a process is under batch, flow, semi-continuous, or continuous flow conditions. In specific embodiments, a process does not include chromatography or chromatographic purification methods. In certain embodiments the process is a cGMP production process.

In some embodiments, this invention provides a sugar-nucleotide prepared by a process according to any of the embodiments of this invention. In preferred embodiments, UDP-galactose is prepared. In certain embodiments, compounds of this invention are obtained from non-animal based plant materials.

Certain embodiments provide a machine configured for a process according to any of the embodiments of this invention. In some embodiments, a process is conducted within one or two reaction vessels.

### BRIEF DESCRIPTION OF THE DRAWINGS

FIG. 1A and FIG. 1B depict a galactose-UDP synthesis flowchart.

FIG. 2 depicts immobilized enzymes and a method of making the same.

FIG. 3A and FIG. 3B depict a galactose-UDP synthesis flowchart employing Tab5.

FIG. 4: Depict a SDS PAGE gel of Tab5 and the sequence of Tab5 SEQ ID NO:1.

FIG. 5 depicts HPLC traces showing the conversion of ADP to AMP and adenosine catalyzed by Tab5 in two different buffers at 4- and 20-hour timepoints.

### DETAILED DESCRIPTION OF THE INVENTION

The invention provides enhanced production efficiency of sugar-nucleotides and related compounds, including but not limited to, galactose containing nucleotides, galactose-containing sugars, and galactose-containing complex carbohydrates while lowering costs. This improves the accessibility and affordability of these molecules. https://www.xenimmune.com/index.html (see panel 3 for UDP-Gal); https://www.dsm.com/human-nutrition/en/products/hmos.html; M. Wicinski et al. Nutrients 12(1):266 (2020); C. A. Autran et al. British Journal of Nutrition 116(2):294-299 (2016); A. Rousseaux, et al. Frontiers in Immunology 12:680911 (2021); D. R. Hill et al. Nutrients 13(10):3364 (2021); M. Zuurveld et al. Frontiers in Immunology 11:801 (2020). The foregoing are incorporated by reference herein in their entirely.

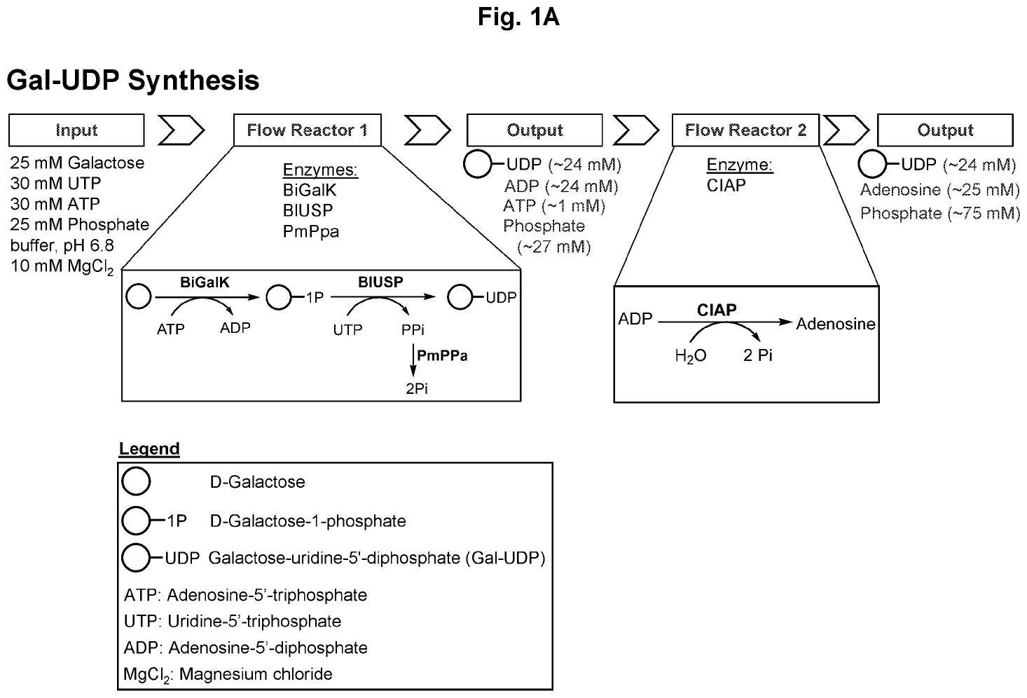

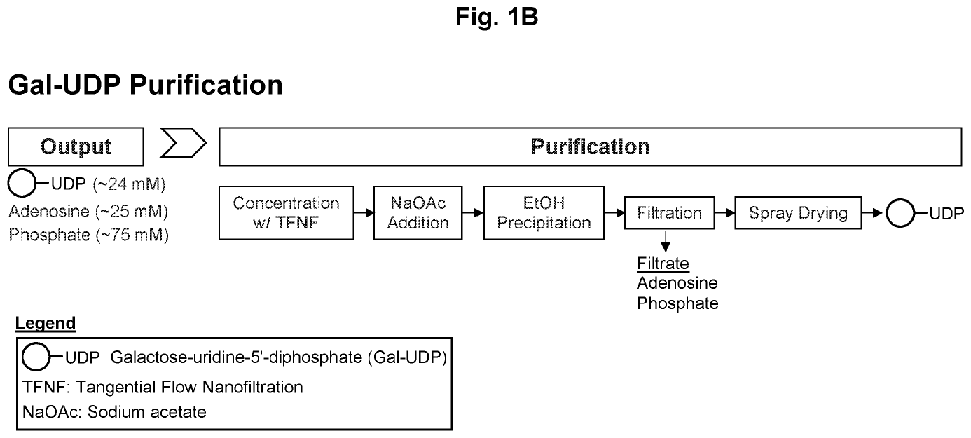

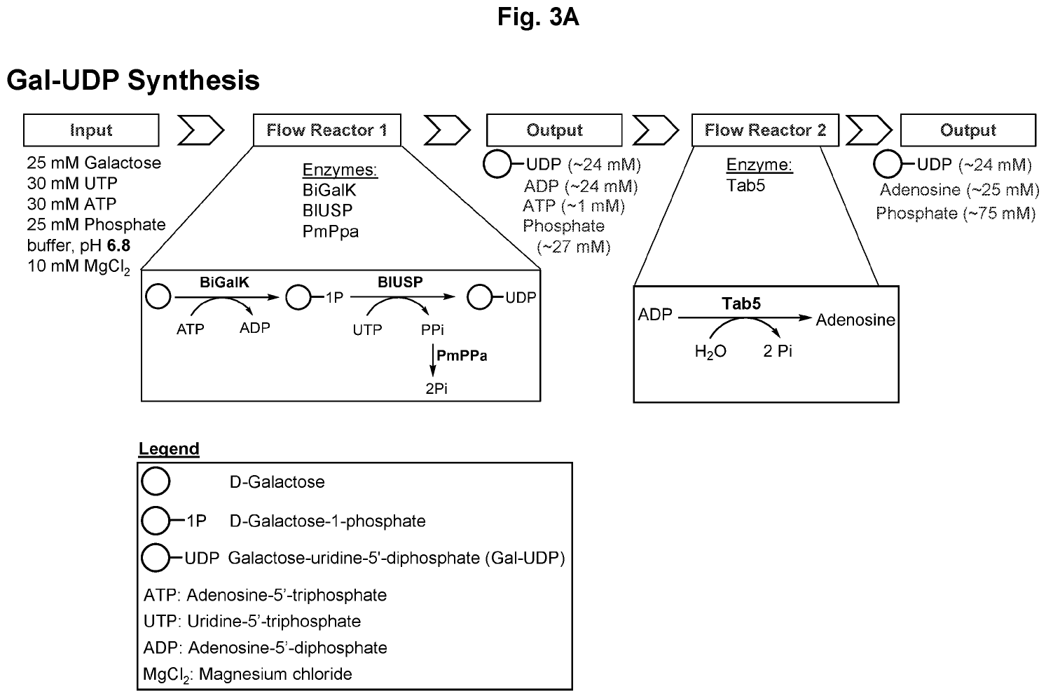

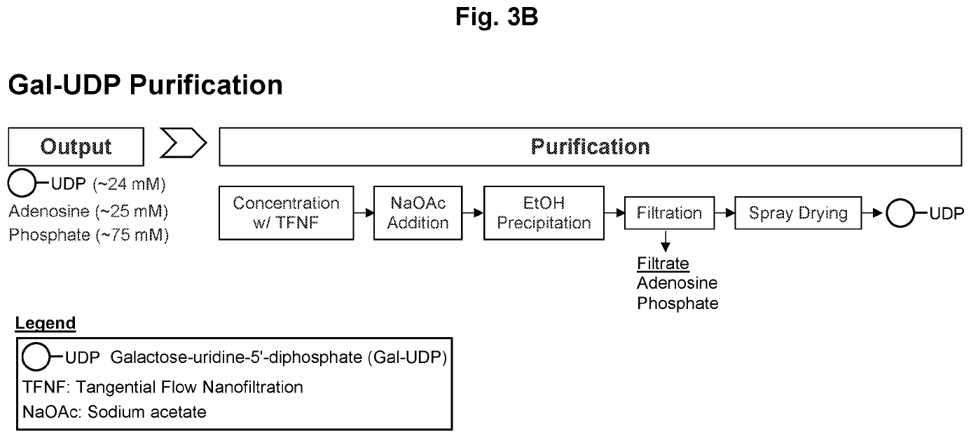

The invention provides processes for obtaining sugar-nucleotide compounds and processes for purifying the obtained sugar-nucleotide compounds. Herein, the terms sugar-nucleotide and nucleotide-sugar are used interchangeably. In one embodiment, the invention provides a process for obtaining a sugar-nucleotide from a reaction mixture comprising a sugar nucleotide and adenosine diphosphate. In one embodiment, the invention provides a process for preparing a sugar-nucleotide and then isolating and purifying the sugar-nucleotide from a reaction mixture. In certain embodiments the process for preparing the sugar-nucleotide is as depicted in FIG. 1A and FIG. 3A and the process for isolating the sugar-nucleotide is a depicted in FIG. 1B and FIG. 3B.

In some embodiments, the invention provides a process for obtaining a sugar-nucleotide from a reaction mixture comprising a sugar nucleotide and adenosine diphosphate. In other embodiments, the invention provides a process for preparing a sugar-nucleotide and then isolating and purifying the sugar-nucleotide from a reaction mixture. In certain embodiments the process for preparing the sugar-nucleotide is as depicted in FIG. 1A and FIG. 3A and the process for isolating the sugar-nucleotide is a depicted in FIG. 1B and FIG. 3B.

In some embodiments this invention provides processes for reducing the amount of adenosine diphosphate in a sugar-nucleotide composition, by using a phosphatase to hydrolyze adenosine diphosphate to adenosine and phosphate under neutral or acidic reaction conditions. Under these conditions, the adenosine diphosphate is hydrolyzed selectively over the sugar-nucleotide. This allows for the preparation a sugar-nucleotide that is substantially free of nucleotide starting materials and nucleotide side-products, including, but not limited to, adenosine triphosphate, and adenosine diphosphate, and adenosine. Enzymatic hydrolysis at higher pH, under basic conditions, results in the hydrolysis of the sugar-nucleotide into a sugar and a nucleotide.

In certain embodiments, the process is provided for separating a sugar-nucleotide and a nucleotide triphosphate, comprising providing a composition comprising a sugar-nucleotide and a nucleotide triphosphate, contacting the composition and a phosphatase at pH 6.9 or lower. In some embodiments, a process is provided for converting adenosine diphosphate to adenosine in the presence of a sugar-nucleotide, comprising providing a composition comprising adenosine diphosphate and a sugar-nucleotide contacting the composition and a phosphatase at pH 6.9 or lower to convert the adenosine diphosphate to adenosine and phosphate.

In certain embodiments, this invention provides a process for converting adenosine diphosphate to adenosine in the presence of a sugar-nucleotide, comprising contacting a phosphatase and a composition comprising adenosine diphosphate and a sugar-nucleotide at acidic pH to convert the adenosine diphosphate to adenosine and phosphate. In other embodiments, is provided a process for preparing a composition comprising adenosine and a sugar-nucleotide, comprising providing a first composition comprising adenosine diphosphate, a sugar-nucleotide, and a phosphatase at pH 6.9 or lower to convert the adenosine diphosphate to adenosine and phosphate to provide a second composition comprising adenosine and a sugar-nucleotide.

Table 1 summarizes the nucleotide and sugar unit combinations that may be prepared according to this invention.

TABLE 1
Nucleotide and Sugar Combinations

| Nucleotide | Sugar Unit |
|---|---|
| UDP | D-Glucose, D-galactose, D-fructose, L-rhamnose, D-mannose, |
| | D-ribose, D-xylose, L-arabinose, D-xylose, L-fucose, |
| | D-apiose, D-glucuronate, D-galacturonate, |
| | N-acetyl-D-glucosamine, N-acetyl-D-galactosamine |
| ADP | D-Glucose, D-galactose, D-fructose, D-mannose, D-ribose, |
| | L-arabinose, D-xylose, D-glucuronate |
| GDP | D-Glucose, D-galactose, D-mannose, L-rhamnose, D-ribose, |
| | D-xylose, L-arabinose, L-fucose |
| CMP | N-acetylneuraminic acid |
| TDP | D-Glucose, D-galacturonate |

In certain embodiments, the sugar-nucleotide is UDP-glucose, UDP-galactose, UDP-fructose, UDP-rhamnose, UDP-mannose, UDP-ribose, UDP-xylose, UDP-arabinose, UDP-xylose, UDP-fucose, UDP-apiose, UDP-glucuronate, UDP-galacturonate, UDP-N-acetyl-D-glucosamine, or UDP-N-acetyl-D-galactosamine.

In certain embodiments, the sugars are in their naturally occurring isomeric forms. In some embodiments, the glucose is D-glucose. In some embodiments, galactose is D-galactose. In some embodiments, fucose is L-fucose. In some embodiments, tagatose is D-tagatose. In some embodiments, N-acetylglucosamine (GlcNAc) is N-Acetyl-D-glucosamine. In some embodiments, fructose is D-fructose.

In certain embodiments, the sugar-nucleotide is UDP-D-Glucose, UDP-D-galactose, UDP-D-fructose, UDP-L-rhamnose, UDP-D-mannose, UDP-D-ribose, UDP-D-xylose, UDP-L-arabinose, UDP-D-xylose, UDP-L-fucose, UDP-D-apiose, UDP-D-glucuronate, UDP-D-galacturonate, UDP-N-acetyl-D-glucosamine, UDP-N-acetyl-D-galactosamine.

In certain embodiments, the sugar-nucleotide is ADP-Glucose, ADP-galactose, ADP-fructose, ADP-mannose, ADP-ribose, ADP-arabinose, ADP-xylose, or ADP-glucuronate, In certain embodiments, the sugar-nucleotide is ADP-D-Glucose, ADP-D-galactose, ADP-D-fructose, ADP-D-mannose, ADP-D-ribose, ADP-L-arabinose, ADP-D-xylose, or ADP-D-glucuronate,

In certain embodiments, the sugar-nucleotide is GDP-Glucose, GDP-galactose, GDP-mannose, GDP-rhamnose, GDP-ribose, GDP-xylose, GDP-arabinose, or GDP-fucose. In certain embodiments, the sugar-nucleotide is GDP-D-Glucose, GDP-D-galactose, GDP-D-mannose, GDP-L-rhamnose, GDP-D-ribose, GDP-D-xylose, GDP-L-arabinose, or GDP-L-fucose.

In certain embodiments, the sugar-nucleotide is CMP-N-acetylneuraminic acid. In certain embodiments, the sugar-nucleotide is TDP-Glucose or TDP-galacturonate. In certain embodiments, the sugar-nucleotide is TDP-D-Glucose or TDP-D-galacturonate.

FIG. 1A and FIG. 1B depict a synthesis route for Gal-UDP according to this invention. Galactose, UTP, ATP, phosphate buffer and MgCl₂ are combined in a flow reactor in the presence of BiGalK, BlUSP, and PmPpa. The enzyme BiGalK in the presence of ATP converts galactose to galactose monophosphate and generates ADP. The enzyme BlUSP converts galactose monophosphate in the presence of UTP to galactose diphosphate and generates pyrophosphate. In the presence of PmPpa, generated pyrophosphate is converted to inorganic phosphate. The reaction mixture output from flow reactor 1 is galactose-UDP, ADP, ATP, and phosphate. The flow reactor 1 output is added to flow reactor 2. In the presence of calf intestine alkaline phosphatase, ADP and water is converted to adenosine and inorganic phosphate. Converting ADP to adenosine facilitates the purification processes of this invention. A nucleoside (here, adenosine) rather than a nucleotide (here, ADP) will stay in solution rather than precipitating along with the sugar-nucleotide in the precipitation step. Thus, unreacted nucleotide is removed prior to precipitation. Accordingly, in FIG. 1A, the flow reactor 2 output is galactose-UDP, adenosine, and phosphate. The flow reactor 2 output mixture is then purified.

First, if free enzymes are used in an embodiment, they are removed prior to the concentration step. If immobilized enzymes are used, they will remain in their respective reaction vessels and will not be present (or be present in small amounts) in the reaction output. If free enzymes are used, they will be present in each reaction's output and will be removed, preferably, prior to product purification. The removal may be accomplished by methods, including but not limited to, ultrafiltration. For clarity, if immobilized enzymes are used, the ultrafiltration step is not required, but may nevertheless be used to ensure any free enzymes are removed. Accordingly, as depicted in FIG. 1B, UDP-galactose is purified by concentrating, adding salt, adding ethanol in a precipitation step, filtration, and spray drying. In certain embodiments, a UDP-galactose process of this invention employs free enzymes a removal step, such as ultrafiltration, is conducted prior to the concentrating. Proteins are retained and removed after Reactor 1 or Reactor 2.

In certain embodiments, processes of this invention involve selective precipitation of the obtained sugar-nucleotides. FIG. 1B depicts the ethanol precipitation employed for the selective precipitation in certain embodiments.

The aqueous product is added into ethanol (EtOH: 90-100% v/v) to selectively precipitate galactose-UDP while keeping the remaining reactants in solution. In certain embodiments the phosphate, MgCl₂, adenosine, and galactose remain in solution and any remaining AMP, ADP, and ATP and unreacted UTP precipitates out of solution. The mixture is filtered and the retentate (galactose-UDP) is spray dried to obtain dry product. The ethanol precipitation step may be repeated to further increase the purity.

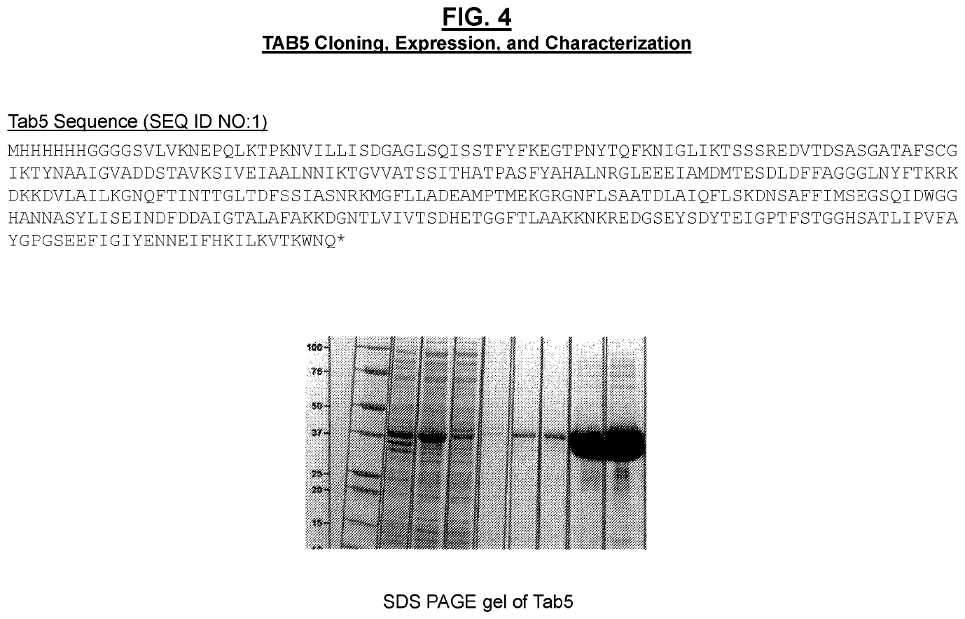

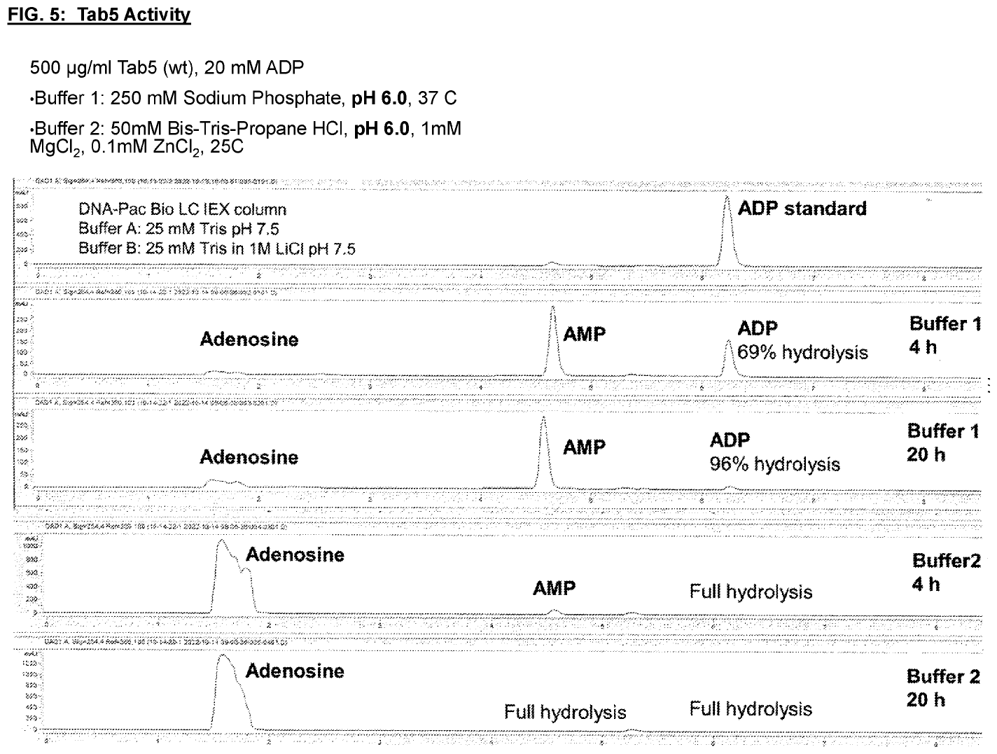

In certain embodiments of this invention, Tab5 is employed to hydrolyze adenosine diphosphate to adenosine and phosphate. FIG. 3A and FIG. 3B depict embodiments of this invention employing Tab5 phosphatase. FIG. 4 relates to Tab5 cloning, expression, and characterization. FIG. 4 depicts the Tab5 sequence used in embodiments of this invention and a SDS PAGE gel of Tab5 demonstrating expression at >95% purity. FIG. 5 depicts a Tab5 activity assessment showing ADP hydrolysis under acidic pH conditions. Percent conversions of ADP to AMP, and qualitative conversion values to adenosine are depicted. Tab5 is a psychrophilic alkaline phosphatase from Antarctic bacterium Tab5 (Uniprot primary accession number Q9KWY4: https://www.uniprot.org/uniprotkb/09KWY4/entry); D. Koutsioulis, et al., Protein Engineering, Design & Selection 21, 5:319-327 (2008); M. Rina, et al., Eur. J Biochem. 267, 1230-1238 (2000); D.-H. Lee, et al., Biotechnology 15, 1 (2015), DOI 10.1186/s12896-015-0115-2. The foregoing are incorporated by reference herein in their entirety. Without being bound by theory, addition of MgCl₂ and/or ZnCl₂ may accelerate hydrolysis (Buffer 2>Buffer 1), and addition in inorganic phosphate may inhibit hydrolysis. See Example 5.

Certain embodiments of this invention provide a process for preparing a composition comprising adenosine and a sugar-nucleotide, comprising providing a first composition comprising adenosine diphosphate, a sugar-nucleotide, and an Antarctic phosphatase, including but not limited to Tab5, to convert the adenosine diphosphate to adenosine and phosphate to provide a second composition comprising adenosine and a sugar-nucleotide.

This invention provides methods for processing, separating, purifying, isolating, concentrating to separate the sugar-nucleotide from the reaction mixture and from adenosine.

In some embodiments, this invention provides a process for preparing a sugar-nucleotide compound, comprising the step of contacting a kinase, an enzyme converting galactose-1-phosphate and UTP to galactose-UDP, an inorganic pyrophosphatase, a sugar, a nucleotide, and ATP to prepare the sugar-nucleotide. In certain embodiments, the process comprises a further step of purifying the sugar-nucleotide.

In some embodiments, this invention provides a process for preparing a sugar-nucleotide compound, comprising the steps of contacting a kinase, an enzyme converting galactose-1-phosphate and UTP to galactose-UDP, an inorganic pyrophosphatase, a sugar, a nucleotide, and ATP to obtain a first output; contacting the first output in the presence of an alkaline phosphatase to obtain a second output; and purifying the second output to obtain the sugar-nucleotide.

In certain embodiments, processes of this invention the sugar-nucleotides are purified on a multi-step process comprising the steps of filtering the second output to remove the enzymes, concentrating the second output to obtain a concentrate, adding a salt the concentrate to obtain a salted concentrate, adding an antisolvent or a water miscible solvent to the salted concentrate to provide a precipitate solution, separating the precipitate solution to obtain a filtrate and a precipitate, drying the precipitate to obtain the nucleotide-sugar. This process is useful if free (non-immobilized) enzymes are employed. Certain embodiments comprise filtering the first output to remove enzymes. Other embodiments comprise filtering the first output and the second output remove enzymes.

In certain embodiments, the filtering step removes enzymes by a filtration or an adsorption method. In certain embodiments the filtration is ultrafiltration, tangential flow filtration, or diafiltration. In specific embodiments, the filtering step is ultrafiltration.

Some embodiments of this invention employ a nucleotide sugar pyrophosphorylase for transferring a nucleotide monophosphate group from a nucleotide triphosphate to a phosphorylated sugar to provide a sugar-dinucleotide. In certain embodiments the nucleotide sugar pyrophosphorylase is a UDP-sugar pyrophosphorylase. In certain embodiments the enzyme converting galactose-1-phosphate and UTP to galactose-UDP is a UDP-sugar pyrophosphorylase (USP) or a UTP-Glucose 1-phosphate uridylyltransferase. In other embodiments, the UDP-sugar pyrophosphorylase is BlUSP, EcUSP, or SpUSP. In certain embodiments the UTP-Glucose 1-phosphate uridylyltransferase is SpGalU. In embodiments depicted in FIG. 1A and FIG. 3A, the nucleotide sugar pyrophosphorylase is BlUSP. M.M. Muthana, et al., Chemical Communications 48.21:2728-2730 (2012) (incorporated by reference herein in its entirety).

Some embodiments of this invention employ a kinase for transferring a phosphate group from ATP to a sugar. In certain embodiments the kinase is a galactokinase. In some embodiments, the galactokinase is BiGalK, SpGalK, EcGalK, or LgGalK. In embodiments depicted in FIG. 1A and FIG. 3A, the kinase is BiGalK. L. Li, et al. “A highly efficient galactokinase from Bifidobacterium infantis with broad substrate specificity” Carbohydrate Research 355:35-39 (2012) (incorporated by reference herein in its entirety).

Some embodiments of this invention employ an inorganic pyrophosphatase for converting inorganic diphosphate to monophosphate. In certain embodiments the inorganic pyrophosphatase is PmPpa or EcPpa. In embodiments depicted in FIG. 1A and FIG. 3A, the inorganic pyrophosphatase is PmPpa. J. Kapyla et al. “Effect of D97E substitution on the kinetic and thermodynamic properties of Escherichia coli inorganic pyrophosphatase” Biochemistry 34.3:792-800 (1995). The foregoing is incorporated by reference herein in its entirety. In the embodiments depicted in FIG. 1B and FIG. 3B, the monophosphate produced is removed from the reaction mixture at the filtration step.

Some embodiments of this invention employ a phosphatase to convert adenosine diphosphate into adenosine and phosphate. In certain embodiments, the phosphatase is an alkaline phosphatase (E.C. 3.1.3.1) or an acidic phosphatase (E.C. 3.1.3.2). In certain embodiments, the reaction mixture containing the phosphatase is at neutral or acidic pH. Without being bound be theory, having non-alkaline reaction conditions allow for ADP to be hydrolyzed to adenosine without substantial amounts of the sugar-nucleotide being hydrolyzed to a sugar and a nucleotide. This aids in isolating and purifying the sugar-nucleotide by allowing adenosine to be separated from the sugar-nucleotide precipitation and filtration methods rather than by chromatographic methods.

In certain embodiments, the alkaline phosphatase is calf intestinal alkaline phosphatase. In other embodiments, the phosphatase is an Antarctic phosphatase. In certain embodiments, the Antarctic phosphatase is Tab5.

In embodiments of this invention employing a phosphatase, the pH is maintained in an acidic range such that ADP is hydrolyzed while minimizing chemical hydrolysis of the sugar-nucleotide, a deleterious nonenzymatic reaction that is catalyzed my magnesium chloride (MgCl₂) and is particularly pronounced at pH>6.9. In some embodiments, the phosphatase reaction (Reactor 2 (two) as depicted in FIG. 1A and FIG. 3A) is at an acidic pH. In some embodiments, the phosphatase reaction is at a pH of precisely, about, at least, up to, or less than, for example, pH 5.0 to pH 6.9. In some embodiments, the phosphate reaction mixture is at pH of precisely, about, at least, up to, or less than, for example, the pH 5, 6, or 7. In some embodiments, the phosphatase reaction mixture is at pH of precisely, about, at least, up to, or less than, for example, the pH 5.0, 5.1, 5.2, 5.3, 5.4, 5.5, 5, 6, 5.7, 5.8, 5.9, 6.0, 6.1, 6.2, 6.3, 6.4, 6.5, 6.6, 6.7, 6.8, or pH 6.9.

In some embodiments, the pH of reactions to obtain sugar-nucleotides (Reactor 1 (one) in FIG. 3A and FIG. 3A) 1 is acidic, neutral, or basic. In certain embodiments, the pH in reactor 1 is precisely or about, for example, between 5.0 and 8.5. The buffer in the input may be phosphate buffer or other buffers that maintain the reaction pH range including, but not limited to PIPES, MOPS, BisTris, Tris, imidazole, sodium phosphate, potassium phosphate, PBS, imidazole, glycine, acetate, citrate, or Good's buffers (including, but not limited to, MES, Bis-tris, PIPES, MOPs, TES, HEPES, TEA, Tris). In some embodiments, acetate is sodium acetate and citrate is sodium citrate.

In certain embodiments, no buffer is used and the pH is continuously adjusted via the addition of concentrated acid, base, or both. In such embodiments, no buffer would be used in Reactor 1 or Reactor 2 as depicted in FIG. 1A and FIG. 3A.

It should be understood that pH may fluctuate during the course of a reaction. Such fluctuations are within the scope of this invention.

Certain embodiments of this invention comprise the steps of filtering the second output to remove enzymes to provide a filtered output composition, concentrating the filtered output composition to obtain a concentrate, adding a salt to the concentrate to obtain a salted concentrate, adding an antisolvent or a water miscible solvent to the salted concentrate to provide a precipitate composition, wherein the precipitate composition comprises a solid phase and a solution phase, separating the precipitate composition to obtain a filtrate and a precipitate, drying the precipitate to obtain the sugar-nucleotide. Certain embodiments comprise the step of filtering the first output composition. Other embodiments comprise the step of filtering the first output composition and the second output composition.

In certain embodiments, this invention employs relatively high reaction concentrations. The use of higher reaction concentrations avoids or minimizes the need to remove water from a reaction mixture. It also allows for smaller reaction vessels to be used, thus decreasing capital expenditures and production costs. In certain embodiments, a process of this invention is conducted at 5 mM to 500 mM galactose. In certain embodiments, a process of this invention is conducted at 250 mM to 500 mM galactose.

In some embodiments, the concentration of adenosine diphosphate in the second composition is 5-100% of the concentration of adenosine diphosphate in the first composition.

In some embodiments, the conversion of adenosine diphosphate to adenosine and phosphate is about 80-100%. In some embodiments, the conversion of adenosine diphosphate to adenosine and phosphate as depicted in Reactor 2 (FIG. 1A and FIG. 3A) is about 80-100%.

In certain embodiments, the sugar-nucleotide is not substantially hydrolyzed to the counterpart sugar and nucleotide based on molar percentages. In certain embodiments, the precisely, about, up to, or less than, for example, 20%, 15%, 10%, or 5% hydrolyzed. In other embodiments, the sugar-nucleotide is precisely, about, up to, or less than, for example, 1%, 2%, 3%, 4%, 5%, 6%, 7%, 8%, 9%, or 10% hydrolyzed. In other embodiments, the sugar-nucleotide is precisely, about, up to, or less than, for example, 0.5% hydrolyzed. In certain embodiments, the sugar-nucleotide as greater than 95% or greater than 99% unhydrolyzed. In some embodiments, the conversion of the sugar-nucleotide into a sugar and a nucleotide precisely or less than about 1%.

In some embodiments, the concentration of the sugar-nucleotide in the second composition is precisely, or at least about 90-100% of the concentration of the sugar-nucleotide in the first composition. In certain embodiments, the concentration of the sugar-nucleotide in the second composition is precisely, at least, or greater than about 95% or precisely, at least, or greater than 99% of the concentration of the sugar-nucleotide in the first composition. In certain embodiments, the concentration of the sugar-nucleotide in the second composition is precisely, at least, or greater than about 0.5% of the concentration of the sugar-nucleotide in the first composition.

In the embodiments depicted in FIG. 1A, FIG. 1B, FIG. 3A, and FIG. 3B, the first composition is the output of Flow Reactor 2 and the second composition is the output of Flow Reactor 1.

This invention provides robust purification methods that do not require chromatography or chromatographic purification methods.

In one embodiment, the invention provides a process for purifying a sugar-nucleotide comprising the steps of concentrating the second output to obtain a concentrate, adding a salt to the concentrate to obtain a salted concentrate, adding a an antisolvent or a water miscible solvent to the salted concentrate to provide a precipitate solution, separating the precipitate solution to obtain a filtrate and a precipitate, and drying the precipitate to obtain the nucleotide-sugar. The precipitate solution comprises a solid phase, comprising the sugar-nucleotide, and a solution phase, comprising the soluble reaction components. The process is useful when immobilized enzymes are employed. In certain embodiments, the concentrate is concentrated by a factor of about or at least 2 to about or at least 100. In other embodiments, the concentrate is concentrated by a factor of about or at least 2 to about or at least 100. In other embodiments, the concentrate is concentrated by a factor of precisely, about, at least, above, up to, or less than, for example, 2, 10, 20, 30, 40, 50, 60, 70, 80, 90, or 100. In certain embodiments, the concentrating is by tangential flow nanofiltration (TFNF).

In certain embodiments the salt added to the concentrate to obtain a salted concentrate is an ammonium salt, a lithium salt, a sodium salt, or a potassium salt. In some embodiments the sodium salt is sodium acetate. In other embodiments, the potassium salt is potassium acetate. In yet other embodiments, the salt is present in an amount of precisely, about, at least, above, up to, or less than, for example, 10, 20, 30, 40, 50, 60, 70, 80, 90, 100, 110, 120, 130, 140, 150, 160, 170, 180, 190, or 200 mM. In certain embodiments, the sodium acetate is present in about 10 mM to about 200 mM. In other embodiments, the potassium acetate is present in about 10 mM to about 200 mM.

In certain embodiments, an antisolvent triggers precipitation of certain reaction mixture components while other components remain in solution. Antisolvents include but are not limited to, ethyl acetate, acetonitrile, tetrahydrofuran, toluene, acetone, pentanol, and octanol. In certain embodiments, antisolvents include, but are not limited to, water miscible solvents. In certain embodiments a water miscible solvent is an alcohol, including, but not limited to an alcohol having 1-8 carbon atoms or an alcohol having 1-6 carbon atoms. In certain embodiments the alcohol is ethanol, methanol, or isopropanol. In other embodiments the alcohol is ethanol.

In embodiments involving an antisolvent or a water miscible solvent, such solvent has a solvent volume in an amount of precisely, about, at least, above, up to, or less than, for example, 2, 3, 4, 5, 6, 7, 8, 9, or 10 times the volume of solvent relative to the salted concentrate volume.

In certain embodiments, the antisolvent or water miscible solvent is incubated with the salted concentrate to provide the precipitate solution. In such embodiments, an incubation is at a temperature of precisely, about, at least, above, up to, or less than, for example −20° C., −15° C., −10° C., −5° C., 0° C. or 4° C. In certain embodiments, the incubation is at a temperature of about −20° C. to about 4° C.

In certain embodiments, the antisolvent or water miscible solvent and the salted concentrate is incubated for precisely, about, at least, above, up to, or less than, for example 2, 4, 6, 8, 10, 12, 14, 16, 18, 20, 22, or 24 hours. In other embodiments, the antisolvent or water miscible solvent and the salted concentrate is incubated for about 2 hours to about 24 hours.

In embodiments of this invention, the separating is by a physical separation method. In certain embodiment, physical separation method is filtration, centrifugation, or decanting.

In embodiments of this invention, the drying is by spray drying, freeze drying, vacuum drying, or evaporative drying. In certain embodiments the drying is by spray drying. The spray-dried sugar-nucleotides are in a salt form based on the salt used herein. In certain embodiments, the sugar-nucleotides are ammonium salts, lithium salts, sodium salts, or potassium salts.

In certain embodiments, the sugar-nucleotide is purified to a level of precisely, about, at least, above, up to, or less than, for example, 80%, 85%, 90%, 95%, or 100%. In certain embodiments, the sugar-nucleotide is precisely, about, at least, above, up to, or less than, for example 85%, 86%, 87%, 88%, 89%, 90%, 91%, 92%, 93%, 94%, 95%, 96%, 97%, 98%, 99%, or 100% free of contaminants, reactants, or reagents used in a process of this invention.

In certain embodiments, UDP-galactose of this invention including by not limited to a spray-dried UDP-galactose is purified to a level of precisely, about, at least, above, up to, or less than, for example, 80%, 85%, 90%, 95%, or 100%. In certain embodiments, the sugar-nucleotide is precisely, about, at least, above, up to, or less than, for example 85%, 86%, 87%, 88%, 89%, 90%, 91%, 92%, 93%, 94%, 95%, 96%, 97%, 98%, 99%, or 100% free of contaminants, reactants, or reagents used in a process of this invention.

In certain embodiments, this invention provides processes for preparing UDP-galactose.

Accordingly, this invention provides a process for preparing UDP-galactose, comprising the step of contacting a galactokinase, a UDP-sugar pyrophosphorylase, an inorganic pyrophosphatase, galactose, UTP, and ATP to prepare UDP-galactose. In certain embodiments, the process comprises a further step of purifying the UDP-galactose.

Accordingly, this invention provides a process for preparing UDP-galactose, comprising the steps of:
a. contacting a galactokinase, a UDP-sugar pyrophosphorylase, an inorganic pyrophosphatase, galactose, UTP, and ATP to form a reaction mixture
b. purifying the reaction mixture to obtain UDP-galactose.

Methods of this invention provide sugar-nucleotides with high purity, of precisely, about, at least, above, or up to, for example 97%, 98%, or 99%. In some embodiments UDP-galactose is obtained that has about or at least 97%, about or at least 98%, or about or at least 99% purity.

The invention provides processes to be carried out in a single reaction vessel to perform one-pot reactions or more than one reaction vessel. The one-pot synthesis may be done in batch or flow, including but not limited to, repetitive batch or continuous flow. In a flow reactor, such as a packed bed reactor, the mixture of reagents is passed through the flow. Thus, in one embodiment, the invention provides a commercially applicable biocatalysis in flow. In in-flow operations, the flow rate is set to control residence time for optimal conversion while maximizing productivity of product production. Embodiments of this invention employing CIAP processes are preferably conducted in a flow reactor.

This invention also provides products, including by not limited to, sugar-nucleotides prepared by any process described herein. In one embodiment, UDP-galactose is prepared by a process of this invention.

This invention also provides a machine configured to carry-out a process of this invention. In certain embodiment, such a process is within a single reaction vessel. The enzymes and reactants used in process of this invention are contacted with each other by combining in a reaction vessel.

In certain embodiments, the sugar-nucleotides of this invention are used for adding a sugar to other compounds, including but not limited to, glycans, carbohydrates, and oligosaccharides, including, but not limited to, two sugar units or more. Thus, sugar containing compounds are synthesized. The synthesis is biocatalytic via employment of a transferase that transfers the activated sugar (sugar-nucleotide) onto an acceptor sugar or other compound.

Sugar nucleotides (e.g., Gal-UDP) can be used to add galactose to sugars. Other sugar nucleotides can be used to make HMOs, including but not limited to LNT, LNnT, and lactose.

In certain embodiments the sugars are plant derived. Accordingly, any of the embodiments of this invention may be employed to obtain a compound from non-animal based plant materials. In certain embodiments, the products obtained from the processes of this invention are not animal derived. Such products are advantageous in markets where animal-free products are desired.

The nucleotides may also be recovered. In the embodiments exemplified herein, UDP, GDP, or ADP may be recovered in galactosylation reactions. In certain embodiments, the nucleotides are converted to the counterpart nucleosides for ease of purification.

Glycans, are carbohydrate-based compounds featuring one or more monosaccharides linked with a glycosidic bond, including N-linked and O-linked bonds. Activated monosaccharides, oligosaccharides, polysaccharides, plant glycans, animal glycans, and microbe glycans are all within the scope of this invention as are glycoconjugates, such as glycolipid, glycopeptides, glycoproteins, and proteoglycans. Glycans also include humanized glycoproteins, humanized antibodies, and glycoconjugate vaccines. C. Reiley, et al. Nature Reviews Nephrology 15(6):346-366 (2019); R. Rappuoli, “Glycoconjugate vaccines: Principles and mechanisms” Science Translational Medicine, 20(456) (2018). The foregoing are incorporated by reference herein in their entirety.

Sugar-nucleotides according to this invention may be used to add a sugar to an acceptor compound. For example, compounds may be galactosylated using the UDP-galactose prepared according to methods of this invention. Such compounds may include, but are not limited to, rare sugars, activated sugars, HMOs, glycans with sugar modifications, glycosylated small molecules, polymerized fiber sugars, inulins, levans, gluconic acid, invert sugar, flavors, and fragrances.

The glycans synthesized in accordance with the invention may be simple or complex glycan that may be linear or branched. Glycans having one or more sugar units are included. In certain embodiments, the glycans have two, three, four, or five sugar units or more units. In some embodiments, the glycans have 1-18 units. In some embodiments, the glycans have 1-10 units. In some embodiments, the glycans have 1-5 units. In certain embodiments, the glycans have 1, 2, 3, 4, 5, 6, 7, 8, 9, 10 units. In certain embodiments, the glycans have 1 unit, 2 units, 3 units, 4 units, 5 units, or 6 units. In some embodiments, the glycans are oligosaccharides. In some embodiments, the glycans are straight chained or branched chained. In certain embodiments, the glycans have 1-6 units and are straight chained. In other embodiments, the glycans have 1-6 units and are branched. In certain embodiments, n is 3, 4, 5, 6, 7, 8, 9, 10, 11, 12, 13, 14, or 15.

Compounds obtained according to this invention, including but not limited to galactosylated compounds, may be used as components in the synthesis of any glycan-containing compound. The materials functionalized with enzymes, or enzyme systems, have applications for the production of pharmaceuticals, biologicals, nutraceuticals, cosmeceuticals, and food ingredients. In certain embodiments, the sugars and oligosaccharides are non-animal derived.

In certain embodiments, the methods provide galactosylated oligosaccharides or galactosylated antibody-glycan conjugates. In preferred embodiments, galactosylated human milk oligosaccharides (HMOs) are produced.

In certain embodiments, the glycan to be reacted (e.g., galactosylated) is 2′-FL, 3′SL, LNTII, LNT, LNnT, LNFPI, LNFPII, LNFPIII, LSTa, LSTb, LSTc, LSTd, DSLNT, LNnH, 3″′₃,3″′₆-di-O-α-Sia-LNnH 3″′₃,3″′₆-di-O-α-Sia-(3″₃,3″₆-di-O-α-Fuc)-LNnH, biantennary sialylated or fucosylated lacto-N-neohexaoses and neoheptaoses, α-2,3-sialyl lacto-N-neopentaose, linear fucosyl- and sialyl-lacto-N-neo-pentaoses, linear lacto-N-neopentaoses, or biantennary lacto-N-neohexaoses and heptaoses. In certain embodiments, sialic acid is obtained from 3′-SL or 6′-SL, including by not limited to, in situ.

The enzymes used herein may be natural or synthetic, bioengineered enzymes, including fusion enzymes. For example, enzymes may be engineered for affinity towards immobilization scaffolds (tags). Enzymes may also be engineered for improved kinetic properties (e.g., lower Km).

In embodiments of this invention, the enzymes are free enzymes or immobilized enzymes. Without being bound by theory, it is assumed that substantially all enzymes in certain embodiments are either free or are immobilized.

In certain embodiments, the enzymes are immobilized. The immobilization of the enzymes may be through covalent immobilization, entrapment, adsorption, molecular tagging with affinity tags, protein affinity tags, noncovalent adsorption, noncovalent deposition, entrapment, physical entrapment, bioconjugation, chelation, cross-linking, disulfide bonds. See, e.g., A. Basso & S. Serban, Mol. Catal. 479: 110607 (2019); Xu et al., Frontiers in Bioeng & Biotech., Published 30 Jun. 2020 publdoi: 10.3389/fbioe.2020.00660; and R. A. Sheldon & S. van Pelt Chem. Soc. Rev., 42:6223 (2013). The foregoing are incorporated by reference herein in their entirety.

In certain embodiments, this invention employs enzymes immobilized using either N-terminal or C-terminal tags including, but not limited to: a. polyarginine (R₆-R₁₅); b. polylysine (K₆-K₁₅); or c. histidine-arginine repeats (HR)₄₋₁₂.

Certain immobilized enzymes, MNPs, macroporous powders, scaffolds, their structures, organizations, suitable enzymes, and uses are described in WO2012/122437, WO2014/055853, WO2016/186879, WO2017/011292, WO2017/180383, WO2018/34877, WO2018/102319, WO2020/051159, WO2020/69227, WO2022/119982, as well as U.S. App. No. 63/285,082, 63/430,271, and 63/448,218. The foregoing are incorporated by reference herein in their entirety.

In embodiments that employ immobilized enzymes, such enzymes include, but are not limited to, enzymes immobilized within bionanocatalysts (BNCs) that in turn are embedded within scaffolds. Bionanocatalysts (BNCs) according to this invention comprise an enzyme self-assembled with magnetic nanoparticles (MNPs). The BNCs self-assemble with the scaffolds.

In some embodiments the immobilized enzymes are non-magnetic. In certain embodiments, such as for oligosaccharides or sugar-nucleotides, to be used in food ingredients, the immobilized enzymes do not comprise nanoparticles.

In other embodiments, the immobilized enzymes involve permanent molecular entrapment of enzymes within self-assembling nanoparticle (NP) clusters. The self-assembly is purely driven by the materials' electrostatic and magnetic interactions. Ionic strength, buffer pH, and NP concentration are the main parameters impacting the immobilization yield and optimized enzyme activity. The clusters are then magnetically templated onto magnetic scaffolds or shapeable magnetic scaffolds.

In these embodiments, immobilized bionanocatalysts are magnetic materials with one or more enzymes that are immobilized and associated with scaffolds. In some embodiments, the scaffolds are high magnetism and high porosity metal oxides or composite blends of thermoplastics or thermosets comprising magnetic particles that form powders. In some embodiments, Selective Laser Sintering (SLS) is used to design and produce objects via 3D printing by sintering composite magnetic powders.

Bionanocatalysts (BNCs) comprise an enzyme self-assembled with magnetic nanoparticles (MNPs). Self-assembled mesoporous aggregate of magnetic nanoparticles comprise a glycan synthesis enzyme, wherein the mesoporous aggregate is immobilized on a magnetic macroporous scaffold. In one embodiment, the immobilized enzymes comprise (i.) a glycan synthesis enzyme self-assembled in magnetic nanoparticles, and (ii.) a magnetic scaffold. A glycan enzyme is immobilized on nanoparticles where the nanoparticles coat a scaffold, and the enzyme is immobilized in or on the mesoporous structure formed by the nanoparticles. In one embodiment, the nanoparticles comprise magnetite (Fe₃O₄) or maghemite (Fe₂O₃). In another embodiment, the nanoparticles comprise a product synthesized from FeCl₂ and FeCl₃, particularly synthesized via continuous coprecipitation of FeCl_{2*4}H₂O and FeCl₃. FeCl₂*4H₂O (Iron (11) chloride tetrahydrate and FeCl₃*6H₂O (Iron (111) chloride hexahydrate. Accordingly, in one embodiment, the nanoparticles comprise magnetite (Fe₃O₄).

A glycan synthesis enzyme is any enzyme that can be used in the synthesis of a glycan. Steps in glycan synthesis may include activating a sugar, transferring a sugar unit thereby extending a sugar, cofactor recycling, and equilibrium shifting. Glycan synthesis enzymes include, but are not limited to, a sugar activation enzyme, a sugar extension enzyme, a reagent regeneration enzyme, a sugar functionalization enzyme, a sugar support enzyme, a sugar removal enzyme.

Some immobilized enzyme materials, and in particular, magnetic materials, for producing glycans use one or more enzymes that are immobilized within bionanocatalysts (BNCs) which in turn are embedded within macroporous scaffolds to provide scaffolded bionanacatalyst (scaffolded BNCs). The scaffolded BNCs may be inside of modular flow cells for flow manufacturing. The modular flow cells may be mixed and matched for a highly customizable and highly efficient manufacturing process. The scaffolded BNCs are used in reactions for synthesizing glycans by contacting a glycan subunit or substrate with a scaffolded BNC to produce a second glycan, contacting a first glycan subunit and a second glycan subunit to produce a glycan comprising the first and second glycan subunits, or contacting a first glycan with a scaffolded BNC to produce a second glycan. Included are processes to modifying a glycan subunit and to connect glycans.

Magnetic enzyme immobilization involves the entrapment of enzymes in mesoporous magnetic clusters that self-assemble around the enzymes (level 1). The immobilization efficiency depends on a number of factors that include the initial concentrations of enzymes and nanoparticles, the nature of the enzyme surface, the electrostatic potential of the enzyme, the nature of the nanoparticle surface, and the time of contact. Enzymes used for industrial purposes in biocatalytic processes should be highly efficient, stable before and during the process, reusable over several biocatalytic cycles, and economical.

Mesoporous aggregates of magnetic nanoparticles may be incorporated into continuous or particulate macroporous scaffolds (level 2). The scaffolds may or may not be magnetic. Such scaffolds are discussed in, e.g., WO2014/055853, WO2017/180383, and S.C. Corgie, et al., Catalysis & Biocatalysis, Chemistry Today 34(5):15-20 (2016), incorporated by reference herein in their entirety. Highly magnetic scaffolds are designed to immobilize, stabilize, and optimize any enzyme. This includes full enzyme systems, at high loading and full activity, and for the production of, e.g., small molecules.

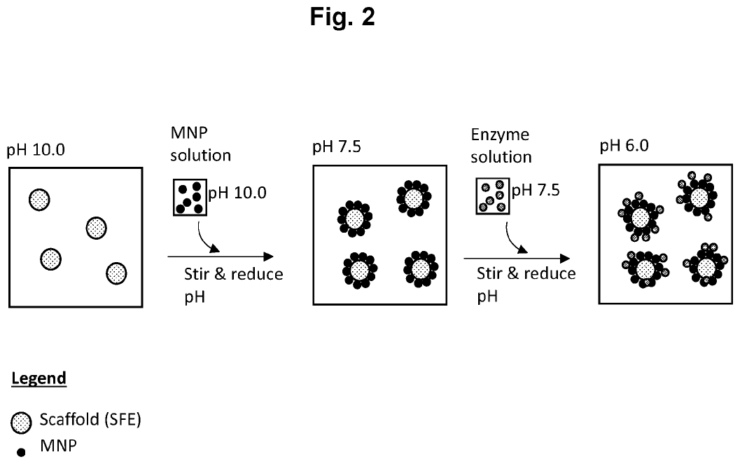

For example, immobilized enzymes may be prepared as depicted in FIG. 2 and employed in process of this invention (WO2022/119982). In a first step, strontium ferrite (SFE) scaffold material is coated with magnetic nanoparticles (MNPs) by lowering the pH from 10.0 to 7.5. In a second step, the glycan synthesis enzymes are added to the product from the first step to the scaffolded BNCs. Type B scaffolded BNC compositions are made by this method.

Certain immobilized enzymes are prepared when glycan synthesis enzymes are contacted with magnetic nanoparticles to form a bionanocatalyst (“BNC”) and then the BNCs are contacted with a magnetic scaffold material. Type A scaffolded BNC compositions are made by this method. In certain embodiments, the magnetic scaffold material is strontium ferrite. In one embodiment the strontium ferrite is a spherical particle with a tight size distribution of an average particle diameter of either 20 μm (S20) or 40 μm (S40W; wrinkled). Strontium ferrite in accordance with this invention available upon request from Powdertech International.

Without being bound by theory, combining enzyme(s) and nanoparticles then adding that combination to the scaffold, the enzymes are entrapped (embedded) within the MNPs (Type A). By adding enzyme(s) to a nanoparticle coated scaffold a Type B composition is obtained, wherein the enzymes remain more exposed at the surface and are not buried as much. As used herein, scaffold-MNP complex, scaffold-MNP matrix, and scaffold-MNP material each indicate the combination of a scaffold and a MNP according to this invention comprising, consisting essentially of, or consisting of, magnetite nanoparticles and a strontium ferrite matrix.

In certain embodiments, this invention employs enzymes immobilized using iron oxide materials including, but not limited to, hematite, magnetite, and strontium ferrite. In certain embodiments the immobilized enzyme is a Type A scaffolded BNC or a Type B scaffolded BNC.

The invention also provides a process for preparing a scaffolded bionanocatalyst by combining a magnetic nanoparticle and a glycan synthesis enzyme to form a bionanocatalyst and then contacting the bionanocatalyst with a scaffold to obtain the scaffolded bionanocatalyst, and a process for preparing a scaffolded bionanocatalyst by combining a scaffold and a magnetic nanoparticle to form a scaffolded magnetic nanoparticle complex and then contacting the scaffolded magnetic nanoparticle complex with a glycan synthesis enzyme. Also provided is a scaffolded BNC made by either of these processes.

Accordingly, this invention provides a glycan synthesis enzyme scaffolded BNC made by the process of contacting strontium ferrite with magnetite nanoparticles to form a scaffold-MNP Complex and adding a glycan synthesis enzyme to the scaffold-MNP Complex to form the scaffolded BNC. Another embodiment provides a glycan synthesis enzyme scaffolded BNC made by the process of combining magnetite nanoparticles and a glycan synthesis enzyme to form a BNC and then contacting the BNC with a scaffold or matrix to form a scaffolded BNC. Without being bound by theory, the bionanocatalyst coats the magnetic microporous scaffold material.

One embodiment of this invention a scaffolded BNC composition comprises a self-assembled mesoporous aggregate of magnetic nanoparticles and a glycan synthesis enzyme and a magnetic microporous material. A scaffolded BNC according to this invention comprises a glycan synthesis enzyme immobilized on magnetic nanoparticles, wherein the magnetic nanoparticles coat a magnetic macroporous material. In certain embodiments, the scaffolded BNCs comprises a magnetic macroporous matrix material comprising self-assembled mesoporous aggregates of magnetic nanoparticles magnetically entrapping an immobilized glycan synthesis enzyme In certain embodiments, the scaffolded BNCs consists of, or consists essentially of, a magnetic macroporous matrix material comprising self-assembled mesoporous aggregates of magnetic nanoparticles magnetically entrapping an immobilized glycan synthesis enzyme. In any embodiments, the scaffolded BNC comprises any elementary enzyme module described herein or is in a system module.

A scaffolded BNC composition comprises a self-assembled mesoporous aggregate of magnetic nanoparticles and a glycan synthesis enzyme and a magnetic microporous material. A scaffolded BNC that may be used in the methods of this invention comprises a glycan synthesis enzyme immobilized on magnetic nanoparticles, wherein the magnetic nanoparticles coat a magnetic macroporous material. In certain embodiments, the scaffolded BNCs consist of a magnetic macroporous matrix material comprising self-assembled mesoporous aggregates of magnetic nanoparticles magnetically entrapping an immobilized glycan synthesis enzyme. In certain embodiments, the scaffolded BNC is used in an integrated pathway as disclosed herein.

Scaffolds according to the invention are chemically inert, structurally tunable to fit any process, and highly magnetic to ensure full capture of the enzyme-containing cluster. In one embodiment, the magnetic macroporous material comprises a metal oxide or a metal oxide complex. In one embodiment, the scaffold comprises a metal oxide. In one embodiment, the metal oxide is strontium ferrite (SrFe₁₂O₁₉). In certain embodiments, the magnetic macroporous material is a metal oxide and consists essentially of, or consists of, metallic materials or ceramic and does not include a polymer. In certain embodiments, the scaffold is a metal oxide and is not a nanoparticle.

The immobilized enzymes provide a series of highly tunable materials and processes for universal enzyme immobilization based on magnetic metamaterials. The unique enzyme hierarchical immobilization platform provides optimal conditions to immobilize single and full systems of enzymes and allows optimal conditions to be found and adapted for single and full systems of enzymes. It affords enzyme stability, maximal use of substrates (including co-factors) and imparts modularity to flow processes. Accordingly, certain methods of this invention use a stabilized enzyme composition comprising a bionanocatalyst and a magnetic scaffold, wherein the bionanocatalyst comprises a glycan synthesis enzyme and magnetic nanoparticles and the magnetic scaffold stabilizes the bionanocatalyst.

One embodiment provides a modular process for producing a glycan, comprising a module that may be a flow cell wherein: the module comprises a magnetic macroporous powder comprising magnetic microparticles, wherein the powder has immobilized a preparation of self-assembled mesoporous aggregates of magnetic nanoparticles containing a glycan synthesis enzyme; wherein a substrate is introduced into the module (or passed through a flow cell) and the substrate is modified to provide a glycan.

Continuous flow reactors including, but not limited to, packed-bed and fixed-bed reactors in tubular format can be combined with upstream and downstream processes that are not continuous making the overall process semi-continuous. For example, the reaction feed for the LNTII reaction (Example 3c-A) may be produced in a continuous stirred tank reactor, the product of which is continuously added to the flow reactor. Microfluidic reactors may also be employed in connection with this invention.

Certain embodiments involve methods comprising 1 or more modules. In a method comprising more than a first module, a first substrate is passed through the first modular flow cell to create a modified substrate; wherein the modified substrate is a second substrate to pass through a second module to create a second modified substrate. The invention provides for sets of modules to be combined allowing the synthesis of complex glycans.

In one embodiment, the invention provides methods for making glycans by immobilizing an enzyme with magnetic nanoparticles and contacting the immobilized enzyme with appropriate synthetic reagents. The methods may be conducted in batch, flow, semi-continuous, or continuous-flow.

The process for preparing the magnetic scaffolds is flexible as employed in this invention provides for convenient, flexible reactions.

The process for preparing the magnetic scaffolds is flexible and tunable to manufacture objects using 3D designs that magnetically capture the BNCs. A large surface area may result from the sintering process itself. Materials can also be recycled by removing the BNCs and then re-functionalized them for repeated use. See PCT/US19/53307, incorporated by reference herein in its entirety.

In some embodiments, thermoplastics are Polyethylene (PE) (varying densities, e.g. LDPE, HDPE), Polypropylene (PP), Acrylics: Polyacrylic acids (PAA), Poly(methyl methacrylate) (PMMA), Polyvinyl alcohol (and polyvinyl acetals), Polyamides (Nylon), Polylactic acid (PLA), Polycarbonate (PC), Polyether sulfone (PES), Polystyrene (PS), Polyvinyl chloride (PVC), Acrylonitrile butadiene styrene (ABS), Polybenzimidazole (PBI), Polyoxymethylene (POM), Polyetherether ketone (PEEK), Polyetherimide (PEI), Polyvinylidene fluoride (PVDF), Polytetrafluoroethylene (PTFE/Teflon), Polyacrylonitrile (PAN)) blended with magnetic materials (e.g. magnetite MMP) via melting/extrusion or via coating of the magnetic material by dissolving the plastic in a solvent. In other embodiments, the powders are sintered by a laser using SLS. Porosity may be formed during SLS.

Selective laser sintering (SLS) is an additive manufacturing (AM) technique that uses a laser as the power source to sinter powdered materials such as plastic, metal, ceramic, glass powders, nylon or polyamide. A laser automatically aimed at points in space, defined by a 3D model (e.g. an Additive Manufacturing File, AMF, or a CAD file), binds the material together to create a solid structure. After each cross-section is scanned, the powder bed is lowered by a one-layer thickness, a new layer of material is applied on top, and the process is repeated until the part is completed. There are many different technologies, such as stereolithography (SLA) or fused deposit modeling (FDM).

SLS is similar to direct metal laser sintering (DMLS) but differs in technical details. DSLM uses a comparable concept, but in DSLM the material is fully melted rather than sintered. This allows one to manufacture materials with different properties (e.g. crystal structure and porosity). SLS is a relatively new technology that may be expanded into commercial-scale manufacturing processes.

In one embodiment, polypropylene-magnetite materials can be 3D-printed in any shape and form via SLS.

In other embodiments, an extruded composite material is size reduced via cryomilling or another form of milling. In other embodiments, composite powders are sieved to an ideal particle size. In preferred embodiments, the particle sizes are 60+/−20 μm.

Powders or 3D printed objects can be functionalized with BNCs containing one or more enzymes or enzyme systems. BNCs are magnetically trapped at the surface of the powders or 3D printed objects.

They may be single-layered or multiple-layered materials that hold the BNCs. Such designed objects may be produced using 3D printing by sintering composite magnetic powders. In some embodiments, Selective Laser Sintering (SLS) is used. The modular flow cells may be mixed and matched for a highly customizable and highly efficient manufacturing process.

Composite powders may also be optimized for flowability. In some embodiments, 3D objects can be printed to optimize flow within to be used in flow reactors.

In some embodiments, 3D objects and composite powders can be washed from the BNCs by an acid wash, rinsed with water, and then re-functionalized with fresh BNCs.

Highly magnetic scaffolds (Macroporous Magnetic Scaffolds or MMP) are designed to immobilize, stabilize and optimize any BNCs containing enzymes. This includes full enzyme systems at high loading and full activity for the production of small molecules. By combining natural or engineered enzymes, and in some embodiments with cofactor recycling systems, the scaffolds allow one to scale up biocatalysis to innovations to manufacturing scale and production.

Included are highly magnetic and highly porous composite blends of thermoplastics with magnetic particles to form powders that may be single-layered or multiple-layered materials that hold the BNCs. Such designed objects may be produced using 3D printing by sintering composite magnetic powders. In some embodiments, Selective Laser Sintering (SLS) is used. The modular flow cells may be mixed and matched for a highly customizable and highly efficient manufacturing process.

MMP made of thermoplastic and magnetic materials of the invention can take the form of magnetic powders that are suitable for flow chemistry application. These powders can be 3D printed by SLS as structures, as functional objects, or as flow cells or plate reactors. High surface areas allow one to maximize the enzyme loading and flow can be engineered within the materials to enable biocatalysis at maximal productivity.

SLS can be used to process nearly any kind of material from metals, ceramics, plastics, and combinations thereof, for tailor-made composite materials. It is critical, however, that the material is available in fine powder form and that the powder particles are operative to fuse when exposing them to heat (J.P. Kruth et al., Assembly Automation 23(4):357-371(2003), incorporated by reference herein in its entirety.

When the material lacks those features, or is prone to phase transitions at the temperature range or conditions of the sintering process, the addition of a sacrificial binder can make this process still feasible for that material. Commonly, polymers are used as sacrificial binders in order to expand the range of materials suitable for this technology. After sintering, the sacrificial binder can be either removed by thermal decomposition or kept as part of the composition.

This concept applies to magnetite that loses its permanent magnetic properties above 585° C. This is significantly lower than its melting temperature (1538° C.). Another advantage of using a polymeric matrix to incorporate magnetite particles is that the former can act as a protective barrier to prevent oxidation and corrosion as well as aiding to disperse the magnetite particles. Also, magnetite can mechanically reinforce the polymer. (Shishkovsky et al., Microelectronic Engineering 146:85-91 (2015), incorporated by reference herein in its entirety).

Laser sintering of plastic parts is one of two additive manufacturing processes used for Rapid Manufacturing (Wegner, Physics Procedia 83:1003-1012 (2016), incorporated by reference herein in its entirety). There are several polymer properties that determine its capability to be sintered and produce good quality 3D objects. These include structural properties such crystalline structure (i.e. thermal properties such as Tm, Tg, and Tc), mechanical properties (Young's modulus and elongation at break, etc.), density, particle size, and shape.

In SLS, the temperature-processing window is determined from the difference between the melting and crystallization temperatures of the polymer. For instance, nylon 12 (PA 12) has one of the highest operational windows and is thus a widely used SLS material. In theory, the higher this value is, the easier the material can be sintered. In practice, however there are many more parameters that can still make this process difficult for any specific polymer (Shishkovsky et al., Microelectronic Engineering 146:85-91 (2015)), incorporated by reference herein in its entirety). In order to prevent curling of the sintered part, a low polymer crystallization rate is desired together with a melt index that provides a suitable rheology and surface tension.

Additionally, the bulk density, particle shape, and size distribution of the powder are key factors (Wegner, Physics Procedia 83:1003-1012 (2016), incorporated by reference herein in its entirety). It has been determined that the in certain embodiments, the optimal particle size range is about 40 to about 90 microns. Smaller particles prevent flowability and their rapid vaporization is detrimental to the optical sensors of the sintering device. This can fog the device and lead to inaccurately sintered parts (Goodridge et al. Materials Science 57:229-267 (2012), incorporated by reference herein in its entirety). The powders should have good flowing properties and preferably an approximately round particle shape. This allows good powder spreading during the process. High heat conductivity of the material is desired at the CO₂ laser beam wavelength (10.6 microns). This is not the case for most polymers. The last two requirements can be met by the incorporation of additives such as high-energy absorption materials, e.g. carbon black, to improve heat absorption, and fume silica nanoparticles (talc) to aid the particle flowability with irregularly-shaped particles.

Additive manufacturing (AM), also referred to as 3D printing, involves manufacturing a part by depositing material layer-by-layer. This differs from conventional processes such as subtractive processes (i.e., milling or drilling), formative processes (i.e., casting or forging), and joining processes (i.e., welding or fastening). Quick production time, low prototyping costs, and design flexibility make 3D printing a valuable tool for both prototyping and industrial manufacturing. The three most common types of 3D printers are fused filament fabrication, stereolithography, and selective laser sintering.

Fused filament fabrication (FFF) melts a thermoplastic continuous filament and builds the object layer by layer until the print is complete. Although alternative materials exist, the two most popular filament materials are polylactic acid (PLA) and acrylonitrile butadiene styrene (ABS). FFF printers and materials are among the cheapest on the market but currently have a lower print resolution and build quality.

Stereolithography (SLA) uses a laser to polymerize photosensitive resins. Uncured liquid resin is placed in a vat where a laser is used to cure resin into solid plastic and build the object layer by layer. SLA printers have a much higher resolution than FFF printers due to the fine spot size of the laser and thus can print intricate features and complex shapes. The resins, however, are more expensive than filaments and completed prints currently require post processing with solvents to optimize the surface finish and material characteristics.

Selective laser sintering (SLS) is a powder-based layer-additive manufacturing process generally meant for rapid prototyping and rapid tooling. Laser beams either in continuous or pulse mode are used as a heat source for scanning and joining powders in predetermined sizes and shapes of layers. The geometry of the scanned layers corresponds to the various cross sections of the computer-aided design (CAD) models or stereolithography (STL) files of the object. After the first layer is scanned, a second layer of loose powder is deposited over it, and the process is repeated from bottom to top until the artifact (3D object) is complete.” Kumar, JOM, 55(10), 43-47 (2003), incorporated by reference herein in its entirety.

SLS provides advantages for printing objects with magnetic properties that can be used for immobilizing BNCs. This is because the printing process creates porosity and a high surface area. The surrounding, unsintered powder acts as a natural support that eliminates the need for dedicated support structures. The lack of support structures allows for complex geometries that would otherwise be impossible to manufacture using alternative 3D printing methods. In addition, the nature of sintering itself creates macro and microporous volumes. During the printing process, the laser flashes thermoplastic crystalline thermoplastic powders (e.g. Polypropylene, polystyrene) between their glass transition temperature and melting temperature to generate stiff parts. By avoiding amorphous behavior with a quick laser scan speed (>100 mm/s), powders are sintered in place to form small bonds amongst themselves. The low-density powders trap air in their structures resulting in remarkable porosity and surface area in three dimensions. These pores increase the surface area for enzyme immobilization.

In recent years, industrial use of enzymes has garnered significant attention due to the wide range of potential manufacturing applications. Using enzymes in industrial processes offers several advantages over conventional chemical methods. This includes high catalytic activity, the ability to perform complex reactions, and promoting greener chemistry by reducing by-products and the need for toxic chemicals (Singh et al., Microbial enzymes: industrial progress in 21st century. 3 Biotech. 6(2):174 (2016), incorporated by reference herein in its entirety).

One of the biggest hindrances to widespread biocatalysis use in industrial production is low enzyme stability. This is further hampered by relatively harsh process conditions that can destabilize enzymes and decrease their lifespan (Mohamad et al., Biotechnology, Biotechnological Equipment 29(2):205-220 (2015), incorporated by reference herein in its entirety). Furthermore, the use of free enzymes in these processes are generally lost from the system as waste products and therefore become a costly operating cost. The primary solution to these issues is immobilization of enzymes onto scaffolding to enhance their operational stability and catalytic activity. Enzyme immobilization also provides a method for enzyme recovery, making biocatalytic processes more economically feasible.

Currently, biocatalytic processes for industrial production are generally carried out in batch reactors due to their simplicity and ease of operation. Despite the benefits of using batch reactors, continuous flow systems enable higher productivity and better process control (Wiles C et al., Green Chem. (14):38-54 (2012)). The rapid development of flow chemistry in biocatalytic processes has primarily been driven by a growing interest in process intensification and green chemistry. Continuous flow systems facilitate process intensification by decreasing residence times (often from hours to minutes), reducing the size of equipment required, and enabling production volume enhancement (Tamborini et al., Cell. 36(1):73-78 (2018)). From a green chemistry standpoint, these systems offer significant improvements in safety, waste generation, and energy efficiency due to heat management and mixing control (Newman and Jensen, Green Chem. (15):1456-1472 (2013)). The foregoing are incorporated by reference in their entirety.

The invention has many benefits over the prior art. It enables the efficient and economical production of glycans, such as complex polysaccharides, including but not limited to, HMOs using enzymes captured in modular flow processing cells. The flow cells may contain materials having large macropores or a high magnetic surface area for BNC immobilization. Flexible compositions for sintered magnetic scaffolds can be made with any meltable thermoplastics and magnetic material composition. The flow cells can have one or multiple enzyme systems that may be pieced together for particular sugar manufacturing processes.

A solution to combining biocatalysis and continuous flow systems is with functionalized flow cells. Biocatalytic flow cells are scaffolds containing immobilized enzymes for use in reactors such as continuous stirred tank reactors (CSTRs) and packed bed reactors (PBRs). Both types of reactors are known in the art but are primarily chosen based on the type of immobilization used. With a total market value of $5.8B in 2010, immobilized enzymes are used in a diverse range of large-scale processes including high fructose corn syrup production (10⁷ tons/year), transesterification of food oils (10⁵ tons/year), biodiesel synthesis (10⁴ tons/year), and chiral resolution of alcohols and amines (10³ tons/year) (R. DiCosimo et al., Chem. Soc. Rev. (42):6437-6474 (2013), incorporated by reference herein in its entirety). These systems allow for improved downstream process management for enzymatic systems compared to batch reactors in terms of in-line control, enzyme reuse, and production scalability.

For the foregoing reasons, the methods of this invention employ biocatalytic systems for small-to-large scale manufacturing using BNCs in scaffolds that are shaped by 3D printing. In some embodiments, the biocatalytic systems are continuous flow.

Scaffolds may comprise cross-linked water-insoluble polymers and an approximately uniform distribution of embedded magnetic microparticles (MMP). The scaffolds may contain thermoset resins including Epoxy resins, Polyesters, Polyurethanes, Melamine resins, Vinyl esters, Silicones (polysiloxanes), Furan resins, Polyurea, Phenolic resins, phenol-formaldehyde, Urea-formaldehyde, Diallyl-phthalate (DAP), Benzoxazine, Polyimides and bismaleimides, Cyanate esters can be used. By combining natural or engineered enzymes, and in some embodiments with cofactors and cofactor recycling systems, the scaffold technology disclosed herein allows one to quickly translate innovation in biocatalysis to innovation in production for batch and flow processes. The magnetic powders are suitable for use flow chemistry applications such as pack-bed reactors.

Self-assembled mesoporous nanoclusters comprising entrapped enzymes are highly active and robust. The technology is a powerful blend of biochemistry, nanotechnology, and bioengineering at three integrated levels of organization: Level 1 is the self-assembly of enzymes with magnetic nanoparticles (MNP) for the synthesis of magnetic mesoporous nanoclusters. This level uses a mechanism of molecular self-entrapment to immobilize and stabilize enzymes. Level 2 is the stabilization of the MNPs into other matrices. Level 3 is product conditioning and packaging for Level 1+2 delivery. The assembly of magnetic nanoparticles adsorbed to enzyme is herein also referred to as a “bionanocatalyst” (BNC). The clusters may be magnetically templated onto shapeable magnetic scaffolds.

MNPs allow for a broader range of operating conditions such as temperature, ionic strength and pH. The size and magnetization of the MNPs affect the formation and structure of the NPs, all of which have a significant impact on the activity of the entrapped enzymes. By virtue of their surprising resilience under various reaction conditions, MNPs can be used as improved enzymatic or catalytic agents where other such agents are currently used. Furthermore, they can be used in other applications where enzymes have not yet been considered or found applicable.

The BNC contains mesopores that are interstitial spaces between the magnetic nanoparticles. The enzymes are preferably embedded or immobilized within at least a portion of mesopores of the BNC. As used herein, the term “magnetic” encompasses all types of useful magnetic characteristics, including permanent magnetic, superparamagnetic, paramagnetic, ferromagnetic, and ferrimagnetic behaviors.

The magnetic nanoparticle or BNC has a size in the nanoscale, i.e., generally no more than 500 nm. As used herein, the term “size” can refer to a diameter of the magnetic nanoparticle when the magnetic nanoparticle is approximately or substantially spherical. In a case where the magnetic nanoparticle is not approximately or substantially spherical (e.g., substantially ovoid or irregular), the term “size” can refer to either the longest the dimension or an average of the three dimensions of the magnetic nanoparticle. The term “size” may also refer to an average of sizes over a population of magnetic nanoparticles (i.e., “average size”).

In different embodiments, the magnetic nanoparticle has a size of precisely, about, up to, or less than, for example, 500 nm, 400 nm, 300 nm, 200 nm, 100 nm, 50 nm, 40 nm, 30 nm, 25 nm, 20 nm, 15 nm, 10 nm, 5 nm, 4 nm, 3 nm, 2 nm, or 1 nm, or a size within a range bounded by any two of the foregoing exemplary sizes.

In the BNC, the individual magnetic nanoparticles can be considered to be primary nanoparticles (i.e., primary crystallites) having any of the sizes provided above. The aggregates of nanoparticles in a BNC are larger in size than the nanoparticles and generally have a size (i.e., secondary size) of at least about 5 nm. In different embodiments, the aggregates have a size of precisely, about, at least, above, up to, or less than, for example, 5 nm, 8 nm, 10 nm, 12 nm, 15 nm, 20 nm, 25 nm, 30 nm, 35 nm, 40 nm, 45 nm, 50 nm, 60 nm, 70 nm, 80 nm, 90 nm, 100 nm, 150 nm, 200 nm, 300 nm, 400 nm, 500 nm, 600 nm, 700 nm, or 800 nm, or a size within a range bounded by any two of the foregoing exemplary sizes.

Typically, the primary and/or aggregated magnetic nanoparticles or BNCs thereof have a distribution of sizes, i.e., they are generally dispersed in size, either narrowly or broadly dispersed. In different embodiments, any range of primary or aggregate sizes can constitute a major or minor proportion of the total range of primary or aggregate sizes. For example, in some embodiments, a particular range of primary particle sizes (for example, at least about 1, 2, 3, 5, or 10 nm and up to about 15, 20, 25, 30, 35, 40, 45, or 50 nm) or a particular range of aggregate particle sizes (for example, at least about 5, 10, 15, or 20 nm and up to about 50, 100, 150, 200, 250, or 300 nm) constitutes at least or above about 50%, 60%, 70%, 80%, 90%, 95%, 98%, 99%, or 100% of the total range of primary particle sizes. In other embodiments, a particular range of primary particle sizes (for example, less than about 1, 2, 3, 5, or 10 nm, or above about 15, 20, 25, 30, 35, 40, 45, or 50 nm) or a particular range of aggregate particle sizes (for example, less than about 20, 10, or 5 nm, or above about 25, 50, 100, 150, 200, 250, or 300 nm) constitutes no more than or less than about 50%, 40%, 30%, 20%, 10%, 5%, 2%, 1%, 0.5%, or 0.1% of the total range of primary particle sizes.

The aggregates of magnetic nanoparticles (i.e., “aggregates”) or BNCs thereof can have any degree of porosity, including a substantial lack of porosity depending upon the quantity of individual primary crystallites they are made of. In particular embodiments, the aggregates are mesoporous by containing interstitial mesopores (i.e., mesopores located between primary magnetic nanoparticles, formed by packing arrangements). The mesopores are generally at least 2 nm and up to 50 nm in size. In different embodiments, the mesopores can have a pore size of precisely or about, for example, 2, 3, 4, 5, 10, 12, 15, 20, 25, 30, 35, 40, 45, or 50 nm, or a pore size within a range bounded by any two of the foregoing exemplary pore sizes. Similar to the case of particle sizes, the mesopores typically have a distribution of sizes, i.e., they are generally dispersed in size, either narrowly or broadly dispersed. In different embodiments, any range of mesopore sizes can constitute a major or minor proportion of the total range of mesopore sizes or of the total pore volume. For example, in some embodiments, a particular range of mesopore sizes (for example, at least about 2, 3, or 5, and up to 8, 10, 15, 20, 25, or 30 nm) constitutes at least or above about 50%, 60%, 70%, 80%, 90%, 95%, 98%, 99%, or 100% of the total range of mesopore sizes or of the total pore volume. In other embodiments, a particular range of mesopore sizes (for example, less than about 2, 3, 4, or 5 nm, or above about 10, 15, 20, 25, 30, 35, 40, 45, or 50 nm) constitutes no more than or less than about 50%, 40%, 30%, 20%, 10%, 5%, 2%, 1%, 0.5%, or 0.1% of the total range of mesopore sizes or of the total pore volume.

The magnetic nanoparticles can have any of the compositions known in the art. In some embodiments, the magnetic nanoparticles are or include a zerovalent metallic portion that is magnetic. Some examples of such zerovalent metals include cobalt, nickel, and iron, and their mixtures and alloys. In other embodiments, the magnetic nanoparticles are or include an oxide of a magnetic metal, such as an oxide of cobalt, nickel, or iron, or a mixture thereof. In some embodiments, the magnetic nanoparticles possess distinct core and surface portions. For example, the magnetic nanoparticles may have a core portion composed of elemental iron, cobalt, or nickel and a surface portion composed of a passivating layer, such as a metal oxide or a noble metal coating, such as a layer of gold, platinum, palladium, or silver. In other embodiments, metal oxide magnetic nanoparticles or aggregates thereof are coated with a layer of a noble metal coating. The noble metal coating may, for example, reduce the number of charges on the magnetic nanoparticle surface, which may beneficially increase dispersibility in solution and better control the size of the BNCs. The noble metal coating protects the magnetic nanoparticles against oxidation, solubilization by leaching or by chelation when chelating organic acids, such as citrate, malonate, or tartrate are used in the biochemical reactions or processes. The passivating layer can have any suitable thickness, and particularly, at least, up to, or less than, about for example, 0.1 nm, 0.2 nm, 0.3 nm, 0.4 nm, 0.5 nm, 0.6 nm, 0.7 nm, 0.8 nm, 0.9 nm, 1 nm, 2 nm, 3 nm, 4 nm, 5 nm, 6 nm, 7 nm, 8 nm, 9 nm, or 10 nm, or a thickness in a range bounded by any two of these values.

Magnetic materials useful in the invention are well-known in the art. Non-limiting examples comprise ferromagnetic and ferromagnetic materials including ores such as iron ore (magnetite or lodestone), cobalt, and nickel. In other embodiments, rare earth magnets are used. Non-limiting examples include neodymium, gadolinium, sysprosium, samarium-cobalt, neodymium-iron-boron, and the like. In yet further embodiments, the magnets comprise composite materials. Non-limiting examples include ceramic, ferrite, and alnico magnets. In preferred embodiments, the magnetic nanoparticles have an iron oxide composition. The iron oxide composition can be any of the magnetic or superparamagnetic iron oxide compositions known in the art, e.g., magnetite (Fe₃O₄), hematite (α-Fe₂O₃), maghemite (γ-Fe₂O₃), or a spinel ferrite according to the formula AB₂O₄, wherein A is a divalent metal (e.g., Xn²+, Ni²+, Mn²⁺, Co²⁺, Ba²⁺, Sr²⁺, or combination thereof) and B is a trivalent metal (e.g., Fe₃+Cr+, or combination thereof).

The individual magnetic nanoparticles or aggregates thereof or BNCs thereof possess any suitable degree of magnetism. For example, the magnetic nanoparticles, BNCs, or BNC scaffold assemblies can possess a saturated magnetization (Ms) of at least or up to about 5, 10, 15, 20, 25, 30, 40, 45, 50, 60, 70, 80, 90, or 100 emu/g. The magnetic nanoparticles, BNCs, or BNC-scaffold assemblies preferably possess a permanent magnetization (Mr) of no more than (i.e., up to) or less than 5 emu/g, and more preferably, up to or less than 4 emu/g, 3 emu/g, 2 emu/g, 1 emu/g, 0.5 emu/g, or 0.1 emu/g. The surface magnetic field of the magnetic nanoparticles, BNCs, or BNC-scaffold assemblies can be about or at least, for example, about 0.5, 1, 5, 10, 50, 100, 200, 300, 400, 500, 600, 700, 800, 900, or 1000 Gauss (G), or a magnetic field within a range bounded by any two of the foregoing values. If microparticles are included, the microparticles may also possess any of the above magnetic strengths.

The magnetic nanoparticles or aggregates thereof can be made to adsorb a suitable amount of enzyme, up to or below a saturation level, depending on the application, to produce the resulting BNC. In different embodiments, the magnetic nanoparticles or aggregates thereof may adsorb about, at least, up to, or less than, for example, 1, 5, 10, 15, 20, 25, or 30 pmol/m2 of enzyme. Alternatively, the magnetic nanoparticles or aggregates thereof may adsorb an amount of enzyme that is about, at least, up to, or less than, for example, about 10%, 20%, 30%, 40%, 50%, 60%, 70%, 80%, 90%, or 100% of a saturation level.

The magnetic nanoparticles or aggregates thereof or BNCs thereof possess any suitable pore volume. For example, the magnetic nanoparticles or aggregates thereof can possess a pore volume of about, at least, up to, or less than, for example, about 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1 cm3/g, or a pore volume within a range bounded by any two of the foregoing values.

The magnetic nanoparticles or aggregates thereof or BNCs thereof possess any suitable specific surface area. For example, the magnetic nanoparticles or aggregates thereof can have a specific surface area of about, at least, up to, or less than, for example, about 50, 60, 70, 80, 90, 100, 110, 120, 130, 140, 150, 160, 170, 180, 190, o r20 0 m 2/g.

The magnetic macroporous matrix material for use according to this invention has a size of precisely, about, up to, or less than, for example, 100-1000, 50-100, 10-50 μm, or 5-10, 1000, 900, 800, 700, 600, 500, 400, 300, 200, 100, 90, 80, 70, 60, 50, 40, 30, 20, 10, 5, less than 5, greater than 100, an average size of 150, an average size of 75, an average size of 40, an average size of 20 or an average size of about 15. In certain embodiments, the material has an average particle diameter of precisely, about, up to, or less than, 20-40 μm, 20 μm, or 40 μm. In certain embodiments, the material has a tight size distribution of an average particle diameter of either 20 μm or 40 μm.

In some embodiments, the methods described herein use recombinant cells that express the enzymes used in the invention. Recombinant DNA technology is known in the art. In some embodiments, cells are transformed with expression vectors such as plasmids that express the enzymes. In other embodiments, the vectors have one or more genetic signals, e.g., for transcriptional initiation, transcriptional termination, translational initiation and translational termination. Here, nucleic acids encoding the enzymes may be cloned in a vector so that they are expressed when properly transformed into a suitable host organism. Suitable host cells may be derived from bacteria, fungi, plants, or animals as is well-known in the art.

Although BNCs (Level 1) provide the bulk of enzyme immobilization capability, they are sometimes too small to be easily captured by standard-strength magnets. Thus, sub-micrometric magnetic materials (Level 2) are used to provide bulk magnetization and added stability to Level 1. Commercially available free magnetite powder, with particle sizes ranging from 50-500 nm, is highly hydrophilic and tends to stick to plastic and metallic surfaces, which, over time, reduces the effective amount of enzyme in a given reactor system. In addition, powdered magnetite is extremely dense, thus driving up shipping costs. It is also rather expensive—especially at particle sizes finer than 100 nm. To overcome these limitations, low-density hybrid materials consisting of magnetite, non-water-soluble cross-linked polymers such as poly(vinylalcohol) (PVA) and carboxymethylcellulose (CMC), have been developed. These materials are formed by freeze-casting and freeze-drying water-soluble polymers followed by cross-linking. These materials have reduced adhesion to external surfaces, require less magnetite, and achieve Level 1 capture that is at least comparable to that of pure magnetite powder.

In one embodiment, the continuous macroporous scaffold has a cross-linked polymeric composition. The polymeric composition can be any of the solid organic, inorganic, or hybrid organic-inorganic polymer compositions known in the art, and may be synthetic or a biopolymer that acts as a binder. Preferably, the polymeric s scaffold does not dissolve or degrade in water or other medium in which the hierarchical catalyst is intended to be used. Some examples of synthetic organic polymers include the vinyl addition polymers (e.g., polyethylene, polypropylene, polystyrene, polyacrylic acid or polyacrylate salt, polymethacrylic acid or polymethacrylate salt, poly(methylmethacrylate), polyvinyl acetate, polyvinyl alcohol, and the like), fluoropolymers (e.g., polyvinylfluoride, polyvinylidenefluoride, polytetrafluoroethylene, and the like), the epoxides (e.g., phenolic resins, resorcinol-formaldehyde resins), the polyamides, the polyurethanes, the polyesters, the polyimides, the polybenzimidazoles, and copolymers thereof. Some examples of biopolymers include the polysaccharides (e.g., cellulose, hemicellulose, xylan, chitosan, inulin, dextran, agarose, and alginic acid), polylactic acid, and polyglycolic acid. In the particular case of cellulose, the cellulose may be microbial- or algae-derived cellulose. Some examples of inorganic or hybrid organic-inorganic polymers include the polysiloxanes (e.g., as prepared by sol gel synthesis, such as polydimethylsiloxane) and polyphosphazenes. In some embodiments, any one or more classes or specific types of polymer compositions provided above are excluded as macroporous scaffolds. In some embodiment the 3D model is an electronic file.

Any of these compositions and methods may be used in the embodiments of this invention to immobilize an enzyme. In some embodiments, the glycan synthesis enzyme is a phosphatase, a kinase, a nucleotide sugar pyrophosphorylase, or an inorganic pyrophosphorylase.

It should be understood that ‘comprise’ is, where context permits, to be interpreted non-exhaustively. Where context permits, each comprise is alternatively “consist essentially of,” or “consist of.”

Some embodiments are as follows.

A process for preparing a sugar-nucleotide compound, comprising the step of contacting a kinase, an enzyme converting galactose-1-phosphate and UTP to galactose-UDP, an inorganic pyrophosphatase, a sugar, a nucleotide, and ATP to prepare the sugar-nucleotide.

The process according to embodiment 1, wherein the process comprises a further step of purifying the sugar-nucleotide.

A process for preparing a sugar-nucleotide compound, comprising the steps of contacting a kinase, an enzyme converting galactose-1-phosphate and UTP to galactose-UDP, an inorganic pyrophosphatase, a sugar, a nucleotide, and ATP to obtain a first output; contacting the first output in the presence of an alkaline phosphatase to obtain a second output; and purifying the second output to obtain the sugar-nucleotide.

The process according to any one of embodiments 1-3, wherein the nucleotide is UDP and the sugar is D-Glucose, D-galactose, D-fructose, L-rhamnose, D-mannose, D-ribose, D-xylose, L-arabinose, D-xylose, L-fucose, D-apiose, D-glucuronate, D-galacturonate, N-acetyl-D-glucosamine, or N-acetyl-D-galactosamine. The process according to any one of embodiments 1-3, wherein the nucleoside is ADP and the sugar is D-Glucose, D-galactose, D-fructose, D-mannose, D-ribose, L-arabinose, D-xylose, or D-glucuronate. The process according to any one of embodiments 1-3, wherein the nucleotide is GDP and the sugar is D-Glucose, D-galactose, D-mannose, L-rhamnose, D-ribose, D-xylose, L-arabinose, or L-fucose. The process according to any one of embodiments 1-3, wherein the nucleotide is CMP and the sugar is N-acetylneuraminic acid. The process according any one of embodiments 1-3, wherein the nucleotide is TDP and the sugar is D-Glucose or D-galacturonate.

The process according to any one of embodiments 2-8, wherein the purifying is by a selective precipitation.

The process according to any one of embodiments 2-8, wherein the purifying comprises the steps of filtering the second output to remove the enzymes, concentrating the second output to obtain a concentrate, adding a salt the concentrate to obtain a salted concentrate, adding a water miscible solvent to the salted concentrate to provide a precipitate solution, separating the precipitate solution to obtain a filtrate and a precipitate, drying the precipitate to obtain the sugar-nucleotide. The process according to embodiment 10 wherein the separating step removes the enzymes by a filtration or an adsorption method. The process according to embodiment 11, wherein the filtration is ultrafiltration, tangential flow filtration, or diafiltration.

The process according to any one of embodiments 10-12, wherein the purifying comprises the steps of concentrating the second output to obtain a concentrate, adding a salt to the concentrate to obtain a salted concentrate, adding a water miscible solvent to the salted concentrate to provide a precipitate solution, separating the precipitate solution to obtain a filtrate and a precipitate, drying the precipitate to obtain the sugar-nucleotide. The process according to embodiment 13, wherein the concentrating to obtain the concentrate is by a factor of about 2 to about 100. The process according to embodiment 13, wherein the concentrating is by tangential flow nanofiltration.

The process according to any one of embodiments 10-15, wherein the salt is an ammonium salt, a lithium salt, a sodium salt, or a potassium salt. The process according to embodiment 16, wherein the sodium salt is sodium acetate or the potassium salt is potassium acetate. The process according to embodiment 17 wherein the sodium acetate is present in about 10 mM to about 200 mM. The process according to embodiment 17, wherein the potassium acetate is present in about 10 mM to about 200 mM.

The process according to any one of embodiments 10-19, wherein the water miscible solvent is an alcohol. The process according to embodiment 20, wherein the alcohol is ethanol, methanol, or isopropanol. The process according to embodiment 21, wherein the alcohol is ethanol. The process according to any one of embodiments 10-22, wherein the water miscible solvent has a solvent volume of 2, 3, 4, 5, 6, 7, 8, 9, or 10 volumes of solvent relative to the salted concentrate volume.

The process according to any one of embodiments 2-23, further comprising an incubation step, wherein the water miscible solvent and the salted concentrate are incubated. The process according to embodiments 24, wherein the incubation step is at an incubation temperature of about −20° C. to about 4° C. The process according to embodiment 24, wherein the incubation step is at an incubation time of about 2 hours to about 24 hours.

The process according to any one of embodiments 2-26, wherein the separating is by a physical separation method. The process according to embodiment 27, wherein the physical separation method is filtration, centrifugation, or decanting.

The process according to any one of embodiments 10-28, wherein the drying is by spray drying, freeze drying, vacuum drying, or evaporative drying. The process according to embodiments 29, wherein the drying is by spray drying.

The process according to any one of embodiments 1-30, wherein the enzyme is a free enzyme.

The process according to any one of embodiments 1-30, wherein the enzyme is an immobilized enzyme. The process according to embodiment 32, wherein the immobilized enzyme is immobilized via a N-terminal tag or a C-terminal tag. The process according to embodiment 33, wherein the N-terminal tag or the C-terminal tag is polyarginine, polylysine, or histidine-arginine repeats. The process according to embodiment 32, wherein the immobilized enzyme is immobilized via an iron oxide material. The process according to embodiment 35, wherein the iron oxide material is hematite, magnetite, or strontium ferrite. The process according to embodiment 36, wherein the immobilized enzyme is a Type A scaffolded BNC. The process according to embodiment 36, wherein the immobilized enzyme is a Type B scaffolded BNC.

The process according to any one of embodiments 1-4 and 9-38, wherein the sugar-nucleotide pyrophosphorylase is a UDP-sugar pyrophosphorylase. The process according to embodiment 39 wherein the enzyme converting galactose-1-phosphate and UTP to galactose-UDP is a UDP-sugar pyrophosphorylase (USP) or a UTP-Glucose 1-phosphate uridylyltransferase. The process according to embodiment 40 wherein the UDP-sugar pyrophosphorylase is BlUSP, EcUSP, or SpUSP. The process according to embodiment 40, wherein the UTP-Glucose 1-phosphate uridylyltransferase is SpGalU.

The process according to any one of embodiments 1-6 or 9-42, wherein the kinase is a galactokinase. The process according to embodiment 43, wherein the galactokinase is BiGalK, SpGalK, EcGalK, or LgGalK.

The process according to any one of embodiments 1-44, wherein the inorganic pyrophosphatase in PmPpa or EcPpa.

The process according to any one of embodiments 3-45, wherein the alkaline phosphatase is calf intestinal alkaline phosphatase.

The process according to any one of embodiments 1-4 or 9-42, wherein the sugar-nucleotide is UDP-galactose. The process according to any one of embodiments 1-4 or 9-47, wherein the UDP-galactose has about or at least 97%, about or at least 98%, or about or at least 99% purity. Spec; further precipitation.

The process according to any one of embodiments 1-48, wherein the process occurs in a single reaction vessel. The process according to any one of embodiments 1-49, wherein the process is under batch, flow, semi-continuous, or continuous flow conditions. The process according to embodiments 50, wherein the process is in a continuous flow reactor.

The process according to any one of embodiments 1-51, wherein the process does not include chromatography or chromatographic purification methods. The process according to any one of embodiments 1-52, wherein the process is a cGMP production process.

A sugar-nucleotide prepared by a process according to any one of embodiments 1-53. UDP-galactose prepared by a process according to any one of embodiments 1-4, or 9-54.

A compound prepared by a process according to any one of embodiments 1-55, wherein the compound is obtained from non-animal based plant materials.

A machine configured for the process of any one of embodiments 1-53. The machine of embodiment 57, wherein the process is within one or two reaction vessels.

In order that the invention described herein may be more fully understood, the following examples are set forth. It should be understood that these examples are for illustrative purposes only and are not to be construed as limiting this invention in any manner.

### Examples

Abbreviations
PBS: Phosphate buffered saline
MES: 2-(N-morpholino)ethanesulfonic acid
Bis-tris: 2-[bis(2-hydroxyethyl)amino]-2-(hydroxymethyl)propane-1,3-diol
PIPES: piperazine-N,N′-bis(2-ethanesulfonic acid)
MOPS: 3-(N-morpholino)propanesulfonic acid
TES: 2-{[1,3-Dihydroxy-2-(hydroxymethyl)propan-2-yl]amino}ethane-1-sulfonic acid
HEPES: 4-(2-Hydroxyethyl)-1-piperazine ethanesulfonic acid
TEA: Triethanolamine
Tris: 2-Amino-2-(hydroxymethyl)propane-1,3-diol
3′-SL: 3′-Sialyllactose
6′-SL: 6′-Sialyllactose
2′-FL: 2′-Fucosyllactose
LNFPI: Lacto-N-fucopentaose I
LNT: Lacto-N-tetraose
LNnT: Lacto-N-neotetraose
LNTII: Lacto-N-triose II
DSLNT: Disialyllacto-N-tetraose
UDP: Uridine 5′-diphosphate
UTP: Uridine 5′-triphosphate
ADP: Adenosine 5′-diphosphate
ATP: Adenosine 5-triphosphate
CMP: Cytidine 5′-monosphate
CDP: Cytidine 5′-diphosphate
CTP: Cytidine 5-triphosphate
GDP: Guanosine 5′-diphosphate
GTP: Guanosine 5-triphosphate
TDP: Thymidine 5′-diphosphate
Tris: Tris(hydroxymethyl)aminomethane
DMC: 2-chloro-1,3-dimethylimidazolinium chloride
Bbhl: β-N-acetylglucosaminidase
GOS: Galactooligosaccharide
Lactose synthase E.C. 2.4.1.22: generates lactose from glucose and UDP-galactose. It consists of galactosyltransferase (B4GALT1 or B4GALT2) and alpha-lactalbumin
USP: UDP-sugar pyrophosphorylase E.C. 2.7.7.64 that catalyzes a reversible transfer of the uridyl group from UTP to sugar-1-phosphate, producing UDP-sugar and pyrophosphate (PPi)
BlUSP: USP from Bifidobacterium longum
EcUSP: USP from Escherichia coli
SpUSP: USP from Streptococcus pneumoniae
UTP-Glucose 1-phosphate uridylyltransferase (GalU) E.C. 2.7.7.9: catalyzes the conversion of glucose-1-phosphate or galactose-1-phosphate to UDP-glucose or UDP-galactose, respectively.
Inorganic pyrophosphatase (Ppa) E.C. 3.6.1.1: catalyzes the hydrolysis of pyrophosphate (PPi) to two monophosphate ions (Pi).
Alkaline phosphatases E.C. 3.1.3.1: catalyzes the hydrolysis of a nucleotide (NMP, NDP, NTP) into the corresponding nucleoside (N) and a phosphate
Acid phosphatases E.C. 3.1.3.2:
GalK: Galactokinase E.C. 2.7.1.6: catalyzes the phosphorylation of galactose to galactose-1-phosphate (Gal-1P) via consumption of one ATP unit.
BiGalK: Galactokinase from Bifidobacterium infantis
SpGalK: Galactokinase from Streptococcus pneumoniae
EcGalK: Galactokinase from Escherichia coli
LgGalK: Galactokinase from Leminorella grimontii
BlUSP: UDP-sugar pyrophosphorylase from Bifidobacterium longum
Ppa: Inorganic pyrophosphatase
PmPpa: Inorganic pyrophosphatase from Pasteurella multocida
EcPpa: Inorganic pyrophosphatase from Escherichia coli
GalU: UTP-glucose 1-phosphate uridylyltransferase
CIAP: Calf intestinal alkaline phosphatase
SpGalU: UTP-glucose-1-phosphate uridylyltransferase

### Example 1: Reaction Specifications

The following conditions may be used in processes to prepare sugar-nucleotides.
1. pH range: 6.0-9.0 for flow reactor 1 (e.g., BiGalK, BlUSP, PmPpa)
2. pH range: 6.0-9.0 for flow reactor 2 (e.g., CIAP)
3. Buffers: sodium phosphate, potassium phosphate, PBS, imidazole, Glycine, Good's buffers (incl. MES, Bis-tris, PIPES, MOPs, TES, HEPES, TEA, Tris).
3. Reagent concentrations:
   a. Galactose: 1-1000 mM
   b. UTP: 1-1.5 molar equivalents relative to galactose (1-1500 mM)
   c. ATP: 1-1.5 molar equivalents relative to galactose (1-1500 mM)
   d. Buffer: 5-250 mM
   e. MgCl₂ or MnCl₂: 0.2-100 mM
   f. USP or GalU enzymes: 0.1-15 mg/ml (either in solution or in packed bed)
   g. GalK enzyme: 0.1-15 mg/ml (either in solution or in packed bed)
   h. Ppa enzyme: 0.1-15 mg/ml (either in solution or in packed bed)
   i. CIAP: 0.1-15 mg/ml (either in solution or in packed bed)
4. Temperature range: 20° C.-70° C.

### Example 2: Downstream Processing Specifications

The following conditions may be used in processes of this invention.
1. Selective precipitation of UDP-Galactose
   a. solvent: alcohols including ethanol, methanol, iso-propanol
   b. solvent volume: 2, 3, 4, 5, 6, 7, 8, 9, 10 volumes of solvent relative to product volume
   c. salt: sodium acetate 10-200 mM, potassium acetate 10-200 mM
   d. incubation temperature: −20° C., to 4° C.
   e. incubation time: 2-24 hrs
2. Removal of enzymes via filtration (ultrafiltration, tangential flow filtration, diafiltration) or other adsorption methods.
3. Separation of precipitate (Galactose-UDP) and solute via filtration, centrifugation, decanting or related technologies.
4. Drying of Galactose-UDP via spray drying, freeze drying, vacuum, or general evaporative methods.

### Example 3. Enzyme Variants to Prepare UDP-Galactose

The following enzymes are used in processes to prepare UDP-galactose.
1. Enzyme converting Galactose-1-phosphate and UTP to Galactose-UDP
   1.1. UDP-sugar pyrophosphorylase (USP) E.C. 2.7.7.64
      1.1.1. BlUSP (Bifidobacterium longum)
      1.1.2. EcUSP (Escherichia coli)
      1.1.3. SpUSP (Streptococcus pneumoniae)
   1.2. UTP-Glucose 1-phosphate uridylyltransferase (GalU) E.C. 2.7.7.9
      1.2.1 SpGalU (Streptococcus pneumoniae)
2. Galactokinase (GalK) E.C. 2.7.1.6
   2.1 BiGalK (Bifidobacterium infantis)
   2.2 SpGalK (Streptococcus pneumoniae)
   2.3 EcGalK (Escherichia coli)
   2.4 LgGalK (Leminorella grimontii)
3. Inorganic pyrophosphatase (Ppa) E.C. 3.6.1.1
   3.1 PmPpa (Pasteurella multocida)
   3.2 EcPpa (Escherichia coli)
4. Phosphoric ester monoester hydrolases E.C. 3.1.3.
   4.1 Alkaline Phosphatases E.C. 3.1.3.1
      4.1.1 Calf Intestinal Alkaline Phosphatase (CIAP)
   4.2 Acid Phosphatases E.C. 3.1.3.2
   4.3 5′-Nucleotidase E.C. 3.1.3.5

### Example 4. In-Flow Synthesis of UDP-Galactose

4a. Reagents and Materials:

The following chemicals reagents are used to synthesize Galactose.UTP: UTP (Uridine 5′-triphosphate trisodium salt; Carbosynth: NU03863), ATP (Adenosine 5′-triphosphate disodium salt hydrate; Carbosynth: NA00135), D-(+)-Galactose (Thermo Scientific: A1281330), Ethanol (200 proof, Fisher BioReagents, BP2818500), Sodium Phosphate Monobasic Dihydrate (Fisher Chemical, S381-3), Sodium Phosphate Dibasic Anhydrous (Fisher Chemical, S375-500) and MgC₂ (Magnesium Chloride; Macron Fine Chemicals: 595804). All water is obtained from a BarnStead Nanopure water purifier (Thermo Scientific, 18.5 MOhm-cm).

Enzymes BiGalK (Bifidobacterium infantis galactokinase), BlUSP (Bifidobacterium longum UDP-sugar pyrophosphorylase) and PmPpa (Pasteurella multocida inorganic pyrophosphatase) are produced in-house recombinantly in E. coli (BL21/DE3). The enzymes are purified from the soluble lysate by affinity chromatography (NiNTA) and the buffer is exchanged by dialyzing against 50 mM Tris pH 7.5. The enzymes are supplemented with 10% (w/w) glycerol and frozen at −80° C. for storage. The plasmids used for protein expression are produced by Genewiz or Genscript by custom synthesis of the insert and splicing into a commercial pET28a vector (Novagen). CIAP is purchased from New England Biolabs (M0525S).

4b. In-Flow Synthesis of Galactose-UDP:

This example describes a process by which D-Galactose (Gal), Uridine-5′-Triphosphate (UTP) and Adenosine-5′-Triphosphate (ATP) is converted in a flow reactor with three co-immobilized enzymes to Galactose-UDP (synonymously referred to as: UDP-Galactose, UDP-Gal; Gal-UDP, Galactose-uridine-5′-diphosphate), Adenosine-5′-diphosphate (ADP) and inorganic phosphate (orthophosphate) (FIG. 1A).

BNCs are formed with 5.0 mg BiGalK, 2.0 mg BlUSP, and 1.0 mg PmPpa in a 100 ml volume of pH 9 water and 25 ml of a pH 9 nanoparticle solution (390 ug/ml). BlUSP, PmPpa and BiGalK each have C-terminal affinity tags consisting of a (HR)4 sequence (Enzyme-GGGGS-HRHRHRHRP*). Scaffolded BNCs are formed by incrementally adding 1 g of strontium ferrite scaffold (Powdertech, cat. no. S20) while mixing with an overhead stirred (500-1000 rpm). The pH is then incrementally lowered to pH 6.0 over the course of an hour. After an additional one hour of incubation, the supernatant is removed, and the scaffolded BNCs are washed with water. The immobilization yield is determined with a Bradford assay. The BNCs are transferred and packed into a column (length 2″, ID 1/16″) which constitute “flow reactor 1”. A syringe pump (New Era Pump Systems) then delivers the reagent stream (100 ml: 25 mM galactose, 30 mM ATP, 30 mM UTP and 10 mM MgCl₂ in 25 mM phosphate pH 6.8 at 37° C.) at 0.5 ml/hr to afford Galactose-UDP at 95% conversion (HPLC). The product is then directly delivered without prior purification via syringe pump (New Era Pump Systems) at 0.5 ml/hr and at a pH of 7.0 through “flow reactor 2” consisting of a BNC with 5.0 mg CIAP which was immobilized as described above.

The flow-through product from the second flow reactor is ˜10-fold concentrated using tangential flow filtration (nanofiltration) and supplemented with 200 mM sodium acetate. Galactose-UDP is precipitated by adding five volumes of ethanol and incubating the solution for 18 hrs at 4° C. The Galactose-UDP precipitate is recovered by centrifugation and spray dried at room temperature.

### Example 5. Tab5 Cloning and Expression

### Tab5 Plasmid and Protein Sequence

Cloning and expression of Tab5, an alkaline phosphatase from the psychrophilic strain TABS (Uniprot: Q9KWY4). The codon optimized Tab5 gene was synthesized GenScript and it was cloned into a pET30a(+) vector using Ndel and Xhol restriction sites while placing a 6His tag at the N-terminus. The final expressible Tab5 sequence (SEQ ID NO:1) was:

MHHHHHHGGGGSVLVKNEPQLKTPKNVILLISDGAGLSQISSTFYFKEGT
PNYTQFKNIGLIKTSSSREDVTDSASGATAFSCGIKTYNAAIGVADDSTA
VKSIVEIAALNNIKTGVVATSSITHATPASFYAHALNRGLEEEIAMDMTE
SDLDFFAGGGLNYFTKRKDKKDVLAILKGNQFTINTTGLTDFSSIASNRK
MGFLLADEAMPTMEKGRGNFLSAATDLAIQFLSKDNSAFFIMSEGSQIDW
GGHANNASYLISEINDFDDAIGTALAFAKKDGNTLVIVTSDHETGGFTLA
AKKNKREDGSEYSDYTEIGPTFSTGGHSATLIPVFAYGPGSEEFIGIYEN
NEIFHKILKVTKWNQ*.

After transformation into BL21(DE3) competent cells, the culture was grown at 37 C in TB media, induced with 0.5 mM IPTG at 18 C and grown for 16 hours.

Purification of Tab5. After completion of expression, the cells were collected by centrifugation and lysed with ultrasonication using a lysis buffer containing 20 mM tris pH 8.0, 150 mM NaCl, 1 mM MgCl₂, 10 mM Imidazole, 10% glycerol, 0.1% Triton X-100. The lysate was filtered, and the enzyme (Tab5) was purified using IMAC NiNTA affinity chromatography. The loading buffer consisted of 20 mM tris pH 8.0, 150 mM NaCl, 1 mM MgCl2, 10 mM Imidazole, 10% glycerol. The wash buffer consisted of 20 mM tris pH 8.0, 300 mM NaCl. 1 mM MgCl2, 25 mM Imidazole, 10% glycerol, and the elution buffer consisted of 20 mM tris pH 8.0, 300 mM NaCl, 1 mM MgCl2, 500 mM Imidazole, 10% glycerol. After elution, the enzyme was dialyzed against 10 mM tris pH 7.4, 1 mM MgCl₂, 10 μM ZnCl₂, and 10% glycerol. 117 mg of Tab5 was collected from a 1 L expression at >95% by SDS-PAGE (FIG. 4).

We conducted a feasibility study for the downstream processing step of flow reactor 2 (FIG. 1A) by testing the propensity of Tab5 to hydrolyze ADP, the principal product of the Gal-UDP reaction of flow reactor 1. In-house purified Tab5 (500 g/m)l was added to 20 mM ADP in either of two buffers and the reaction was run at 37° C. and 25° C., respectively:
Buffer 1: 250 mM Sodium Phosphate, pH 6.0.
Buffer 2: 50 mM Bis-Tris-Propane HCl, pH 6.0, 1 mM MgCl₂, 0.1 mM ZnCl₂ (New England Biolabs (NEB) Antarctica Phosphatase buffer).

Conversion from ADP to AMP and Adenosine was analyzed at 4- and 20-hour timepoints using HPLC (Agilent 1100, Diode array detector, λ=254 nm; DNA-Pac Bio LC IEX column, Buffer A: 25 mM Tris pH 7.5, Buffer B: 25 mM Tris in 1M LiCl pH 7.5). Percent conversions of ADP to AMP, and qualitative conversion values to Adenosine are shown in FIG. 5.

All publications and patent documents disclosed or referred to herein are incorporated by reference herein in their entirety.

The foregoing description has been presented only for purposes of illustration and description. This description is not intended to limit the invention to the precise form disclosed. It is intended that the scope of the invention be defined by the claims appended hereto.

What is claimed:

1. A process for converting adenosine diphosphate to adenosine, comprising the step of contacting a first composition comprising adenosine diphosphate and a sugar-nucleotide with a phosphatase at an acidic pH to convert said first composition to a second composition comprising adenosine, phosphate, and said sugar-nucleotide.

2. The process according to claim 1, further comprising the initial step of generating said first composition by contacting a sugar, a nucleotide triphosphate, and adenosine triphosphate with a kinase, a nucleotide sugar pyrophosphorylase, and an inorganic pyrophosphorylase.

3. The process according to claim 1 or claim 2, wherein said phosphatase is an alkaline or acidic phosphatase.

4. The process according to claim 3, wherein said phosphatase is an Antarctic phosphatase.

5. The process according to claim 4, wherein said Antarctic phosphatase is a bacterial Antarctic phosphatase.

6. The process according to claim 5, wherein said bacterial Antarctic phosphatase has Tab5 activity and has at least a 90% sequence identity to SEQ ID NO:1.

7. The process according to claim 6, wherein said Tab5 has the sequence of SEQ ID NO:1.

8. The process according to any one of claims 1-7, wherein said acidic pH is 6.9 or below.

9. The process according to claim 8, wherein said acidic pH is 5.0 to pH 6.9.

10. The process according to any one of claims 1-9 comprising separating said sugar-nucleotide and said adenosine.

11. The process according to claim 10, wherein said separating is by selective precipitation of said sugar-nucleotide in the presence of an antisolvent.

12. The process according to any one of claims 1-11, further comprising the steps of concentrating said second composition to obtain a concentrate, adding a salt to said concentrate to obtain a salted concentrate, adding an antisolvent to said salted concentrate to provide a precipitate solution, separating said precipitate solution to obtain a filtrate and a precipitate, drying said precipitate to obtain said sugar-nucleotide.

13. The process according to any one of claim 1-12, further comprising an initial step of removing said phosphatase, said kinase, said nucleotide sugar pyrophosphorylase, and said inorganic pyrophosphorylase from said first composition or said second composition.

14. The process according to claim 13 wherein said removing is by a filtration or an adsorption method.

15. The process according to claim 14, wherein said filtration is ultrafiltration, tangential flow filtration, or diafiltration.

16. The process according to any one of claims 12-15, wherein said concentrating to obtain said concentrate is by a factor of about 2 to about 100.

17. The process according to claim 16, wherein said concentrating is by tangential flow nanofiltration.

18. The process according to any one of claims 12-17, wherein said salt is an ammonium salt, a lithium salt, a sodium salt, or a potassium salt.

19. The process according to claim 18, wherein said sodium salt is sodium acetate or said potassium salt is potassium acetate.

20. The process according to claim 19 wherein said sodium acetate is present in about 10 mM to about 200 mM.

21. The process according to claim 19, wherein said potassium acetate is present in about 10 mM to about 200 mM.

22. The process according to any one of claims 11-21, wherein said antisolvent is an alcohol.

23. The process according to claim 22, wherein said alcohol is ethanol, methanol, or isopropanol.

24. The process according to claim 23, wherein said alcohol is ethanol.

25. The process according to any one of claims 11-24, wherein said antisolvent has a solvent volume of 2, 3, 4, 5, 6, 7, 8, 9, or 10 volumes of solvent relative to said salted concentrate volume.

26. The process according to any one of claims 11-25, further comprising an incubation step, wherein said antisolvent and said salted concentrate are incubated.

27. The process according to claim 26, wherein said incubation step is at a temperature of about −20° C. to about 4° C.

28. The process according to claim 26 or claim 27, wherein said incubation step is at an incubation time of about 2 hours to about 24 hours.

29. The process according to any one of claims 10-28, wherein said separating is by a physical separation method.

30. The process according to claim 29, wherein said physical separation method is filtration, centrifugation, or decanting.

31. The process according to any one of claims 12-30, wherein said drying is by spray drying, freeze drying, vacuum drying, or evaporative drying.

32. The process according to claim 31, wherein said drying is by spray drying.

33. The process according to any one of claims 1-32, wherein said nucleotide is uridine 5′-diphosphate (UDP) and said sugar is D-glucose, D-galactose, D-fructose, L-rhamnose, D-mannose, D-ribose, D-xylose, L-arabinose, D-xylose, L-fucose, D-apiose, D-glucuronate, D-galacturonate, N-acetyl-D-glucosamine, or N-acetyl-D-galactosamine.

34. The process according to any one of claims 1-32, wherein said nucleotide is adenosine 5′-diphosphate (ADP) and said sugar is D-glucose, D-galactose, D-fructose, D-mannose, D-ribose, L-arabinose, D-xylose, or D-glucuronate.

35. The process according to any one of claims 1-32, wherein said nucleotide is guanosine 5′-diphosphate (GDP) and said sugar is D-Glucose, D-galactose, D-mannose, L-rhamnose, D-ribose, D-xylose, L-arabinose, or L-fucose.

36. The process according to any one of claims 1-32, wherein said nucleotide is cytidine 5′-monosphate (CMP) and said sugar is N-acetylneuraminic acid.

37. The process according any one of claims 1-32, wherein said nucleotide is thymidine 5′-diphosphate (TDP) and said sugar is D-Glucose or D-galacturonate.

38. The process according to any one of claims 1-37, wherein said phosphatase, said kinase, said nucleotide sugar pyrophosphorylase, or said inorganic pyrophosphorylase is a free enzyme.

39. The process according to any one of claims 1-37, wherein said phosphatase, said kinase, said nucleotide sugar pyrophosphorylase, or said inorganic pyrophosphorylase is an immobilized enzyme.

40. The process according to claim 39, wherein said immobilized enzyme is immobilized via a N-terminal tag or a C-terminal tag.

41. The process according to claim 40, wherein said N-terminal tag or said C-terminal tag comprises polyarginine, polylysine, or histidine-arginine repeats.

42. The process according to claim 39, wherein said immobilized enzyme is immobilized via an iron oxide material.

43. The process according to claim 42, wherein said iron oxide material is hematite, magnetite, or strontium ferrite.

44. The process according to claim 42 or claim 43, wherein said immobilized enzyme is a Type A scaffolded BNC.

45. The process according to to claim 42 or claim 43, wherein said immobilized enzyme is a Type B scaffolded BNC.

46. The process according to any one of claims 2-45, wherein said sugar-nucleotide pyrophosphorylase is a uridine 5′-diphosphate-sugar-pyrophosphorylase (UDP-sugar pyrophosphorylase).

47. The process according to claim 46 wherein said nucleotide sugar pyrophosphorylase is a UDP-sugar pyrophosphorylase (USP) or a UTP-glucose 1-phosphate uridylyltransferase (GalU).

48. The process according to claim 47 wherein said UDP-sugar pyrophosphorylase is BlUSP, EcUSP, or SpUSP.

49. The process according to claim 47, wherein said UTP-Glucose 1-phosphate uridylyltransferase is SpGalU.

50. The process according to any one of claims 2-49, wherein said kinase is a galactokinase.

51. The process according to claim 50, wherein said galactokinase is galactokinase from Bifidobacterium infantis (BiGalK), galactokinase from Streptococcus pneumoniae (SpGalK), galactokinase from Escherichia coli (EcGalK), or galactokinase from Leminorella grimontii (LgGalK).

52. The process according to any one of claims 2-51, wherein said inorganic pyrophosphatase is inorganic pyrophosphatase from Pasteurella multocida (PmPpa) or inorganic pyrophosphatase from Escherichia coli (EcPpa).

53. The process according to any one of claims 1-52, wherein said sugar-nucleotide is UDP-galactose.

54. The process according to claim 53, wherein said UDP-galactose has about or at least 97%, about or at least 98%, or about or at least 99% purity.

55. The process according to any one of claims 1-54, wherein said process occurs in a single reaction vessel.

56. The process according to any one of claims 1-55, wherein said process is under batch, flow, semi-continuous, or continuous flow conditions.

57. The process according to claim 56, wherein said process is in a continuous flow reactor.

58. The process according to any one of claims 1-57, wherein said process does not include chromatography or chromatographic purification methods.

59. The process according to any one of claims 1-58, wherein said process is a cGMP production process.

60. A sugar-nucleotide prepared by a process according to any one of claims 1-59.

61. UDP-galactose prepared by a process according to any one of claims 1-33 or claims 38-59.

62. A compound prepared by a process according to any one of claims 1-59, wherein said compound is obtained from non-animal based plant materials.

63. A machine configured for said process of any one of claims 1-59.

64. The machine according to claim 63, wherein said process is within one or two reaction vessels.
